SEQUENCE LISTING (46 sequences)

SEQ ID NO: 1 - protein, 13 aa, synthetic construct
  REGION 1..13: note = Peptide of invention comprising 13 amino acids
  SITE 1: note = Leu, Val or Ile, or a non-natural or natural analogue thereof
  SITE 2: note = Val, or a non-natural or natural analogue thereof
  SITE 3: note = Val, or a non-natural or natural analogue thereof
  SITE 4: note = natural or non-natural amino acid
  SITE 5: note = natural or non-natural amino acid
  SITE 6: note = natural or non-natural amino acid
  SITE 7: note = Tyr, or a non-natural or natural analogue thereof
  SITE 8: note = Leu, Val, or Ile, or a non-natural or natural analogue thereof
  SITE 9: note = Pro, or a non-natural or natural analogue thereof
  SITE 10: note = natural or non-natural amino acid
  SITE 11: note = natural or non-natural amino acid
  SITE 12: note = natural or non-natural amino acid
  SITE 13: note = Asp or Glu, or a non-natural or natural analogue thereof
XVVXXXYXPXXXX

SEQ ID NO: 2 - protein, 14 aa, synthetic construct
  REGION 1..14: note = Peptide of invention comprising 14 amino acids
  SITE 1: note = Leu, Val or Ile, or a non-natural or natural analogue thereof
  SITE 2: note = Val, or a non-natural or natural analogue thereof
  SITE 3: note = Val, or a non-natural or natural analogue thereof
  SITE 4: note = natural or non-natural amino acid
  SITE 5: note = natural or non-natural amino acid
  SITE 6: note = natural or non-natural amino acid
  SITE 7: note = Tyr, or a non-natural or natural analogue thereof
  SITE 8: note = Leu, Val, or Ile, or a non-natural or natural analogue thereof
  SITE 9: note = Pro, or a non-natural or natural analogue thereof
  SITE 10: note = natural or non-natural amino acid
  SITE 11: note = natural or non-natural amino acid
  SITE 12: note = natural or non-natural amino acid
  SITE 13: note = Asp or Glu, or a non-natural or natural analogue thereof
  SITE 14: note = natural or non-natural amino acid
XVVXXXYXPXXXXX

SEQ ID NO: 3 - protein, 13 aa, synthetic construct
  REGION 1..13: note = Reference peptide with 13 amino acids
LVVSTTYLPHYFD

SEQ ID NO: 4 - protein, 13 aa, synthetic construct
  REGION 1..13: note = Peptide
VVSTTYLPHYFDN

SEQ ID NO: 5 - protein, 14 aa, synthetic construct
  REGION 1..14: note = Reference peptide with 14 amino acids
LVVSTTYLPHYFDN

SEQ ID NO: 6 - protein, 14 aa, synthetic construct
  REGION 1..14: note = Peptide
VVVSTTYLPHYFDN

SEQ ID NO: 7 - protein, 14 aa, synthetic construct
  REGION 1..14: note = Peptide
IVVSTTYLPHYFDN

SEQ ID NO: 8 - protein, 14 aa, synthetic construct
  REGION 1..14: note = Peptide
  SITE 1: note = Abu
XVVSTTYLPHYFDN

SEQ ID NO: 9 - protein, 14 aa, synthetic construct
  REGION 1..14: note = Peptide
  SITE 3: note = Chg: 2-amino-2-cyclohexylacetic acid
LVXSTTYLPHYFDN

SEQ ID NO: 10 - protein, 14 aa, synthetic construct
  REGION 1..14: note = Peptide
  SITE 7: note = TYR3-I
LVVSTTXLPHYFDN

SEQ ID NO: 11 - protein, 14 aa, synthetic construct
  REGION 1..14: note = Peptide
  SITE 1: note = Chg: 2-amino-2-cyclohexylacetic acid
  SITE 2: note = Cpa: cyano-propionic amino acid
  SITE 7: note = TYR3,5-I2
XXVSTTXLPHYFDN

SEQ ID NO: 12 - protein, 14 aa, synthetic construct
  REGION 1..14: note = Peptide
  SITE 1: note = Chg: 2-amino-2-cyclohexylacetic acid
  SITE 2: note = Nle
  SITE 3: note = Chg: 2-amino-2-cyclohexylacetic acid
XXXSTTYLPHYFDN

SEQ ID NO: 13 - protein, 17 aa, synthetic construct
  REGION 1..17: note = Peptide
ETALVVSTTYLPHYFDN

SEQ ID NO: 14 - protein, 16 aa, synthetic construct
  REGION 1..16: note = Peptide
TALVVSTTYLPHYFDN

SEQ ID NO: 15 - protein, 15 aa, synthetic construct
  REGION 1..15: note = Peptide
ALVVSTTYLPHYFDN

SEQ ID NO: 16 - protein, 16 aa, synthetic construct
  REGION 1..16: note = Peptide
ETALVVSTTYLPHYFD

SEQ ID NO: 17 - protein, 231 aa, synthetic construct
  REGION 1..231: note = Reference peptide
QVQLVQSGAEVKKPGSSVMVSCQASGGPLRNYIINWLRQAPGQGPEWMGGIIPVLGTVHYAPKFQGRVTITADESTDTAYIHLISLRSEDTAMYYCATETALVVSTTYLPHYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 18 - protein, 12 aa, synthetic construct
  REGION 1..12: note = Peptide
VSTTYLPHYFDN

SEQ ID NO: 19 - protein, 11 aa, synthetic construct
  REGION 1..11: note = Peptide
STTYLPHYFDN

SEQ ID NO: 20 - protein, 10 aa, synthetic construct
  REGION 1..10: note = Peptide
TTYLPHYFDN

SEQ ID NO: 21 - protein, 9 aa, synthetic construct
  REGION 1..9: note = Peptide
TYLPHYFDN

SEQ ID NO: 22 - protein, 8 aa, synthetic construct
  REGION 1..8: note = Peptide
YLPHYFDN

SEQ ID NO: 23 - protein, 7 aa, synthetic construct
  REGION 1..7: note = Peptide
LPHYFDN

SEQ ID NO: 24 - protein, 15 aa, synthetic construct
  REGION 1..15: note = Peptide
ETALVVSTTYLPHYF

SEQ ID NO: 25 - protein, 14 aa, synthetic construct
  REGION 1..14: note = Peptide
ETALVVSTTYLPHY

SEQ ID NO: 26 - protein, 13 aa, synthetic construct
  REGION 1..13: note = Peptide
ETALVVSTTYLPH

SEQ ID NO: 27 - protein, 12 aa, synthetic construct
  REGION 1..12: note = Peptide
ETALVVSTTYLP

SEQ ID NO: 28 - protein, 10 aa, synthetic construct
  REGION 1..10: note = Peptide
ETALVVSTTY

SEQ ID NO: 29 - protein, 9 aa, synthetic construct
  REGION 1..9: note = Peptide
ETALVVSTT

SEQ ID NO: 30 - protein, 8 aa, synthetic construct
  REGION 1..8: note = Peptide
ETALVVST

SEQ ID NO: 31 - protein, 7 aa, synthetic construct
  REGION 1..7: note = Peptide
ETALVVS

SEQ ID NO: 32 - protein, 14 aa, synthetic construct
  REGION 1..14: note = Peptide
AVVSTTYLPHYFDN

SEQ ID NO: 33 - protein, 14 aa, synthetic construct
  REGION 1..14: note = Peptide
LAVSTTYLPHYFDN

SEQ ID NO: 34 - protein, 14 aa, synthetic construct
  REGION 1..14: note = Peptide
LVASTTYLPHYFDN

SEQ ID NO: 35 - protein, 14 aa, synthetic construct
  REGION 1..14: note = Peptide
LVVATTYLPHYFDN

SEQ ID NO: 36 - protein, 14 aa, synthetic construct
  REGION 1..14: note = Peptide
LVVSATYLPHYFDN

SEQ ID NO: 37 - protein, 14 aa, synthetic construct
  REGION 1..14: note = Peptide
LVVSTAYLPHYFDN

SEQ ID NO: 38 - protein, 14 aa, synthetic construct
  REGION 1..14: note = Peptide
LVVSTTALPHYFDN

SEQ ID NO: 39 - protein, 14 aa, synthetic construct
  REGION 1..14: note = Peptide
LVVSTTYAPHYFDN

SEQ ID NO: 40 - protein, 14 aa, synthetic construct
  REGION 1..14: note = Peptide Figure 4
LVVSTTYLAHYFDN

SEQ ID NO: 41 - protein, 14 aa, synthetic construct
  REGION 1..14: note = Peptide
LVVSTTYLPAYFDN

SEQ ID NO: 42 - protein, 14 aa, synthetic construct
  REGION 1..14: note = Peptide
LVVSTTYLPHAFDN

SEQ ID NO: 43 - protein, 14 aa, synthetic construct
  REGION 1..14: note = Peptide
LVVSTTYLPHYADN

SEQ ID NO: 44 - protein, 14 aa, synthetic construct
  REGION 1..14: note = Peptide
LVVSTTYLPHYFAN

SEQ ID NO: 45 - protein, 14 aa, synthetic construct
  REGION 1..14: note = Peptide
LVVSTTYLPHYFDA

SEQ ID NO: 46 - protein, 19 aa, synthetic construct
  REGION 1..19: note = Peptide
CETALVVSTTYLPHYFDNC

(19) United States
(12) Patent Application Publication
(10) Pub. No.: US 2025/0057802 A1
(43) Pub. Date: Feb. 20, 2025

(54) BROAD-SPECTRUM ANTIVIRAL DRUGS

(71) Applicant: FRAUNHOFER-GESELLSCHAFT ZUR FÖRDERUNG DER ANGEWANDTEN FORSCHUNG E.V., München (DE)

(72) Inventors: Thomas GRUNWALD, Leipzig (DE); Leila ISSMAIL, Lepzig (DE); Daniel RAMSBECK, Halle (Saale) (DE); Martin KLEINSCHMIDT, Halle (Saale) (DE); Christian JÄGER, Halle (Saale) (DE); Mirko BUCHHOLZ, Halle (Saale) (DE); David SMITH, Leipzig (DE); Christin MÖSER, Gräfenhainichen (DE)

(21) Appl. No.: 18/577,179

(22) PCT Filed: Jul. 8, 2022
(86) PCT No.: PCT/EP2022/069087

§ 371 Date: Jan. 5, 2024

(30) Foreign Application Priority Data
Jul. 8, 2021 (EP) 21184516.9

(51) Int. Cl. A61K 31/235; A61K 31/166; A61K 31/36; A61K 38/08; A61K 38/10; A61K 47/54; A61P 31/14

(52) U.S. Cl. CPC A61K 31/235; A61K 31/166; A61K 31/36; A61K 38/08; A61K 38/10; A61K 47/549; A61P 31/14

(57) ABSTRACT

The present invention generally relates to broad-spectrum antiviral compounds that can be used as preventive and/or therapeutic antiviral medicine; pharmaceutical compositions or combinations comprising such compounds; and compounds or pharmaceutical compositions or combinations for use in the treatment (e.g., for preventive and/or therapeutic use) of viral infections.

### FIELD OF THE INVENTION

The present invention generally relates to broad-spectrum antiviral compounds that can be used as preventive and/or therapeutic antiviral medicine; pharmaceutical compositions or combinations comprising such compounds; and compounds or pharmaceutical compositions or combinations for use in the treatment (e.g., for preventive and/or therapeutic use) of viral infections.

### BACKGROUND ART

Viral infections are one of the major causes of morbidity and mortality worldwide; and continue to threaten global public health causing a significant impact on society and economy. There is still a large group of infectious viruses for which no efficient vaccine or therapy has been approved.

A virus of great significance is the respiratory syncytial virus (RSV), also referred to as Human orthopneumovirus, which can lead to serious respiratory complications in premature infants, immunocompromised patients and elderly. RSV is an enveloped negative-sense single-stranded RNA virus of the family Pneumoviridae (genus Orthopneumovirus). RSV has a double layer lipid envelope, in which glycoproteins are embedded, including a Fusion (F) glycoprotein and an Adhesion (G) glycoprotein. There are two subgroups of RSV, A and B, which differ in the structure of the antigen of the Adhesion (G) glycoprotein. The Adhesion (G) glycoprotein is responsible for the specific adsorption of the virus particle onto the cell surface. During the entry of the virus into a host cell, the Fusion (F) glycoprotein induces fusion of viral and cellular membranes leading to delivery of the nucleocapsid into the cytoplasm. The Fusion (F) glycoprotein further interacts directly with heparan sulfate and may participate in virus attachment to the host cell.

RSV leads to infections of the respiratory tract, especially affecting the mucosa of the upper airways as well as the ciliated epithelium of the trachea and bronchia. Virus replication takes place in the ciliated epithelial cells of the mucous membranes of the respiratory tract. The epithelial cells are reversibly damaged by syncytia formation caused by the Fusion (F) glycoprotein and the body's own immune reaction.

Despite research efforts, there is neither a preventive vaccine nor an efficient treatment against RSV infection, and no effective, low-cost prophylactic or therapeutic approach has been developed. Many attempts for the development of effective vaccines or antibodies against RSV failed to confer protection either at preclinical or clinical stages. The only commercially available product is the monoclonal antibody Palivizumab (Synagis®, approved in 1998), the use of which is limited to high-risk patients in industrialized countries due to its high cost and the need of repetitive administrations. Palivizumab is used exclusively for seasonal immunoprophylaxis of RSV infection in populations with high risk for severe illness including premature infants and those suffering from chronic pulmonary disease. Palivizumab has limitations in clinical applications due to its high costs (Five doses of Palivizumab for a 5 kg infant cost approximately $5600) and the need for repeated administrations (IAN (1998): Palivizumab, a Humanized Respiratory Syncytial Virus Monoclonal Antibody, Reduces Hospitalization From Respiratory Syncytial Virus Infection in High-risk Infants. PEDIATRICS 102 (3), pp. 531-537; Homaira et al. (2014): Effectiveness of Palivizumab in Preventing RSV Hospitalization in High Risk Children: A Real-World Perspective. International journal of pediatrics Volume 2014, Article ID 571609).

The treatment of RSV involves mainly symptom management and supportive care. Inhaled corticosteroids might be helpful in reducing RSV associated bronchiolitis symptoms. Ribavirin, a nucleoside analogue that blocks viral replication, is prescribed as a treatment in severe cases of RSV infection, but has little or no significant effect on reduction of RSV load. Besides, ribavirin is expensive, has teratogenic effects in animals and cannot be taken in pregnancy (Krilov, Leonard R. (2002): Safety issues related to the administration of ribavirin. In The Pediatric infectious disease journal 21 (5), pp. 479-481). Thus, unmet medical need for novel clinical and cost-effective antiviral agents against RSV remains a significant problem.

There is also unmet demand for clinically applicable and cost-effective antiviral agents against further viruses, such as Herpes simplex virus 1 (HSV-1), Human papillomavirus (HPV), the Severe acute respiratory syndrome coronavirus 2 (SARS-CoV-2), and Zika virus (ZIKV).

Besides the known viral strains with no available antiviral therapy, new strains emerge that might have epidemic potential and can rapidly spread worldwide leading to global pandemics. For other new emerging viruses, treatment is based mainly on supportive and symptomatic management whereas the prevention of infection depends solely on limiting the contact with infected individuals. Thus, broad-spectrum antivirals are of a great significance for fast combating of emerging viral infections.

Although some plant-derived antiviral compounds have been identified in the past, their therapeutic or prophylactic application has been compromised by poor activity and/or poor chemical stability.

To address these issues, there is demand for improved antiviral drugs having broad antiviral activity spectrum against different viral pathogens and allowing the prophylaxis and/or treatment of severe viral infections.

### Problems Underlying the Invention

The present invention aims at providing compounds having potent antiviral activity. Preferably, the compounds should have broad-spectrum efficacy, including high inhibitory activity against Riboviria, more preferably Negarnaviricota (such as RSV), Pisuviricota (such as SARS-CoV-2), or Flavivirus (such as ZIKV); and/or further viruses, including Duplodnaviria (such as HSV-1) or Monodnaviria (such as HPV). Preferably, the compounds should exhibit low toxicity levels, to enable the treatment or prophylaxis against viral infections (such as RSV infection) in high-risk population, and/or may exhibit high stability.

### SUMMARY OF THE INVENTION

As a solution, the present invention provides broad-spectrum antiviral compounds that can be used as preventive and/or therapeutic antiviral medicine; pharmaceutical compositions or combinations comprising such compounds; and compounds or pharmaceutical compositions or combinations for use in the treatment (e.g., for preventive and/or therapeutic use) of viral infections.

Specifically, the present invention provides compound, or a pharmaceutical composition comprising the compound, for use in a method for treating a viral infection caused by a virus belonging to a taxon selected from Duplodnaviria; Monodnaviria; Negarnaviricota; Pisuviricota; and Flavivirus; wherein the compound has a structure according to Formula (I), an enantiomer, a diastereoisomer, a hydrate, solvate, a crystal form, a tautomer, or a pharmaceutically acceptable salt thereof:

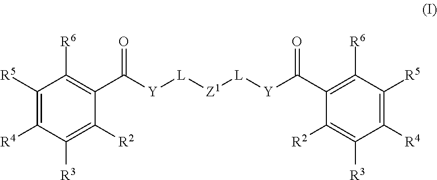

wherein:
L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
Y is selected, independently for each occurrence, from O and NR⁷;
Z¹ is selected from bond, O, NR, CH₂, CH(R), CH(R¹), and CR(R¹);
R is selected from OH, optionally substituted C₁₋₆ alkyl, NR⁷R⁸, and

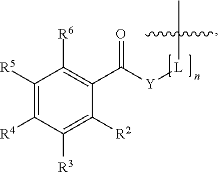

wherein n is 0 or 1;
R¹ is selected from OH, optionally substituted C₁₋₆ alkyl, and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, or R⁶ is OH, and/or wherein at least two occurrences of R², R³, R⁴, R⁵, or R⁶ form together part of an optionally substituted 4- to 8-membered heterocyclic ring containing O; and
R⁷ and R⁸ are selected, independently for each occurrence, from H and optionally substituted C₁₋₆ alkyl.

The present invention also provides a compound, or a pharmaceutical composition comprising the compound, for use in a method for treating a viral infection, wherein the compound has a structure according to the above Formula (I), an enantiomer, a diastereoisomer, a hydrate, a solvate, a crystal form, a tautomer, or a pharmaceutically acceptable salt thereof; and wherein in Formula (I):
L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
Y is selected, independently for each occurrence, from O and NR⁷;
Z¹ is selected from bond, O, CH(R), CH(R¹), and CR(R¹);
R is selected from OH, optionally substituted C₁₋₆ alkyl, NR⁷R⁸, and

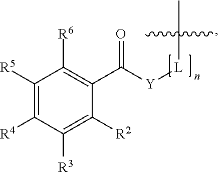

wherein n is 0 or 1;
R¹ is selected from OH, optionally substituted C₁₋₆ alkyl, and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, or R⁶ is OH, and/or wherein at least two occurrences of R², R³, R⁴, R⁵, or R⁶ form together part of an optionally substituted 4- to 8-membered heterocyclic ring containing O; and
R⁷ and R⁸ are selected, independently for each occurrence, from H and optionally substituted C₁₋₆ alkyl.

The present invention also provides a compound, or a pharmaceutical composition comprising the compound, wherein the compound has a structure according to the above Formula (I), an enantiomer, a diastereoisomer, hydrate, a solvate, a crystal form, a tautomer, or a pharmaceutically acceptable salt thereof, and wherein in Formula (I):
L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
Y is selected, independently for each occurrence, from O and NR⁷;
Z¹ is selected from bond, CH(R), CH(R¹), and CR(R¹); wherein if all occurrences of Y are O, then Z¹ is selected from bond, CH(R¹), and CR(R¹);
R is selected from OH, optionally substituted C₁₋₆ alkyl, NR⁷R⁸, and

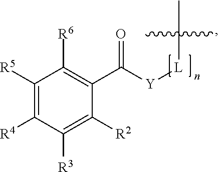

wherein n is 0 or 1;
R¹ is selected from optionally substituted C₁₋₆ alkyl and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence R², R³, R⁴, R⁵, and R⁶ is OH, and/or wherein at least two occurrences of R², R³, R⁴, R⁵, and R⁶ form together part of an optionally substituted 4- to 8-membered heterocyclic ring containing O; and
R⁷ and R⁸ are selected, independently for each occurrence, from H and optionally substituted C₁₋₆ alkyl,
wherein the compound does not have the structure:

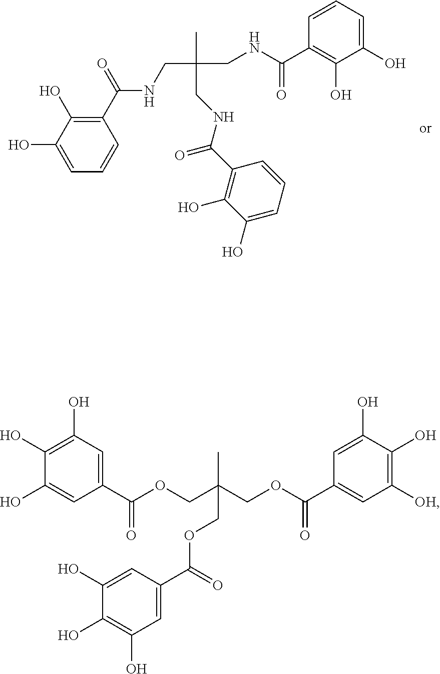 [SMILES: CC(CNC(=O)c1cccc(O)c1O)(CNC(=O)c1cccc(O)c1O)CNC(=O)c1cccc(O)c1O ; CC(COC(=O)c1cc(O)c(O)c(O)c1)(COC(=O)c1cc(O)c(O)c(O)c1)COC(=O)c1cc(O)c(O)c(O)c1]

The present invention also provides a pharmaceutical combination comprising: a compound having a structure according to the above Formula (I), an enantiomer, a diastereoisomer, hydrate, a solvate, a crystal form, a tautomer, or a pharmaceutically acceptable salt; and a peptide or a nanostructure;

the peptide having:
a length of 25 amino acids or less and comprising the sequence X₁—X₂—X₃—X₄—X₅—X₆—X₇—X₈—X₉—X₁₀—X₁₁—X₁₂—X₁₃ (SEQ ID NO: 1), wherein X₁ is Leu, Val or Ile, or a non-natural or natural analogue thereof, X₂ is Val, or a non-natural or natural analogue thereof, X₃ is Val, or a non-natural or natural analogue thereof, X₄ is a natural or non-natural amino acid, X₅ is a natural or non-natural amino acid, X₆ is a natural or non-natural amino acid, X₇ is Tyr, or a non-natural or natural analogue thereof, X₈ is Leu, Val, or Ile, or a non-natural or natural analogue thereof, X₉ is Pro, or a non-natural or natural analogue thereof, X₁₀ is a natural or non-natural amino acid, X₁₁ is a natural or non-natural amino acid, X₁₂ is a natural or non-natural amino acid, X₁₃ is Asp or Glu, or a non-natural or natural analogue thereof, and wherein at least one amino acid in the sequence pursuant to SEQ ID No: 1 is different from the amino acid in the sequence LVVSTTYLPHYFD (SEQ ID No: 3) at the corresponding position, or a peptidomimetic or retro-inverso peptide thereof; or
a length of 25 amino acids or less and comprising the sequence X₁—X₂—X₃—X₄—X₅—X₆—X₇—X₈—X₉—X₁₀—X₁₁—X₁₂—X₁₃ (SEQ ID NO: 2), wherein X₁ is Leu, Val or Ile, or a non-natural or natural analogue thereof, X₂ is Val, or a non-natural or natural analogue thereof, X₃ is Val, or a non-natural or natural analogue thereof, X₄ is a natural or non-natural amino acid, X₅ is a natural or non-natural amino acid, X₆ is a natural or non-natural amino acid, X₇ is Tyr, or a non-natural or natural analogue thereof, X₈ is Leu, Val, or Ile, or a non-natural or natural analogue thereof, X₉ is Pro, or a non-natural or natural analogue thereof, X₁₀ is a natural or non-natural amino acid, X₁₁ is a natural or non-natural amino acid, X₁₂ is a natural or non-natural amino acid, X₁₃ is Asp or Glu, or a non-natural or natural analogue thereof, X₁₄ is a natural or non-natural amino acid, and wherein at least one amino acid in the sequence pursuant to SEQ ID No: 2 is different from the amino acid in the sequence LVVSTTYLPHYFDN (SEQ ID No: 5) at the corresponding position,
or a peptidomimetic or retro-inverso peptide thereof;
the nanostructure comprising:
   a) a nucleic acid scaffold; and
   b) at least two peptide moieties, wherein the sequence of each of the at least two peptide moieties is independently selected from the sequence of a peptide as specified above, wherein the nucleic acid scaffold is selected from the group of:
   i) a linear nucleic acid scaffold, wherein the at least two peptide moieties are each attached at or near different ends of the nucleic acid scaffold; and
   ii) a branched nucleic acid scaffold, wherein the at least two peptide moieties are each attached to a different branch of the scaffold.

### BRIEF DESCRIPTION OF THE DRAWINGS

FIG. 1 to 6 show dose-response curves for identified inhibitors against RSV and in vitro cytotoxicity analysis.

FIG. 7 shows the results of an experiment concerning the mechanism of inhibition of RSV infection.

FIG. 8 shows broad-spectrum in vitro antiviral activity of identified small molecule inhibitors.

FIG. 9 shows antiviral activity of small molecule compounds against SARS-CoV-2 using cell culture-based infection model.

FIG. 10 shows results of stability tests of small molecule compounds in solution.

FIG. 11 shows antiviral activity against RSV of (A) a small molecule inhibitor (23); and (B) combination of a peptide (peptide 6) and a small molecule inhibitor (23).

### DETAILED DESCRIPTION OF THE INVENTION

To design have identified improved antiviral compounds with unexpected antiviral potency. The designed compounds provide an attractive opportunity for potent and cost-effective antiviral therapies against viral infections, such as RSV infection and many other viral diseases.

Plant-based compounds with antiviral activity play an important role in the development of clinically useful antivirals and offer a source for the discovery of novel antiviral compounds. Plant-derived dicaffeoylquinic acid (DCQAs) compounds have been investigated for their antiviral activity. DCQAs showed strong inhibitory effects against RSV with low cytotoxicity in cell culture assays. Moreover, prophylactic treatment of mice with DCQAs lead to a reduction of viral loads in the lungs. However, the poor chemical stability of DCQA in the biological environment makes it rather unsuitable for clinical application. Additionally, there is a need for compounds with high antiviral activity against one or multiple viruses.

The present inventors have identified small-molecule compounds having high potential as effective broad-spectrum antiviral agents against a wide range of viruses, particularly RSV, SARS-CoV-2, HSV-1, HPV, and ZIKV. The compounds may have multiple advantages, including one or more of the following:
Potent inhibitory activity against RSV in vitro and low toxicity levels, which makes them promising candidates for the treatment or prophylaxis against RSV infection in high-risk population.
Broad-spectrum efficacy against other viruses, including SARS-CoV-2, HSV-1, HPV, and ZIKV.
The broad-spectrum antiviral compounds might be useful as a fast and cost-effective intervention approach against novel and emerging viruses.
The compounds offer a prophylactic approach when no vaccine is available or for population groups for which the benefits of vaccination are limited such as immunocompromised patients and elderly.
The compounds can be highly useful as a therapy of viral respiratory co-infections and can be used in a combination with other antivirals to achieve additive effects.

The compounds have potent antiviral activity against RSV as well as multiple other viruses including, but not limited to, SARS-CoV-2, HSV-1, ZIKV and HPV. The compounds may act directly on the viral particle, rather than on the target cells. These compounds may serve as cost-effective prophylactic and/or therapeutic broad-acting antiviral agents, and may be used either alone or in combinations with other viral inhibitors to treat viral single- or co-infections. Moreover, the antiviral compounds disclosed herein provide an opportunity for fast interventions against medically relevant emerging viruses.

The compounds may be used in combination with a peptide or nanostructure as further described below. The peptides show in vitro and in vivo antiviral efficacy. It has been demonstrated that the intranasal delivery of these peptides results in significant reduction of viral load in infected mice. A neutralizing epitope (Ø) may be chosen as a target and a computational method was used to design several peptide inhibitors of RSV fusion; the designed peptides bind specifically to the Ø site of the prefusion conformation of F protein and prevent its transition into the postfusion state. After binding, F protein cannot perform its fusion function anymore, and thus RSV infection of the host cell can be blocked.

Said peptides offer a variety of advantages. The mechanism of antiviral action of peptides involves preventing the fusion of RSV with target cell membrane by capturing RSV—F protein in its metastable prefusion conformation, thus making them suitable for RSV prophylaxis. Further, the targeting of RSV surface glycoprotein (F) by the peptides reduces undesirable side-effects. Moreover, short peptide sequences in the peptides offer a simpler and less expensive production opportunity than antibodies or proteins. Also, the peptides are safer and less toxic than synthetic molecules, and peptides are more stable than antibodies at different storage conditions. Further, the demonstrated in vivo efficacy after intranasal administration allows for non-invasive delivery of peptides directly to the site of action. This will minimize side-effects, decrease required doses and make the application outside hospital settings possible. In addition, the peptides can be used in a combination with small-molecule inhibitor compounds to achieve synergetic effects.

To increase binding efficacy, the peptides can be conjugated to nucleic acid nanostructures of the invention, such as DNA nanostructures. Those nanostructures may function as scaffold material and in contrast to common antibodies, the number of binding sites (peptides) and distances between them may be variable. Since RSV—F is a trimeric surface protein, a three-armed DNA nanostructure is preferably be chosen and modified with three new RSV—F binding peptides, but also other arrangements are possible. Since the persistence length of DNA may be 50 nm, the arms can be stiff. However, the center junction may be flexible and varied by incorporating unpaired bases, for example 3 or 5 extra thymines. In addition, the attachment of such peptides to DNA may improve their solubility and stability against proteases. In embodiments, to enhance their antiviral activity, three peptides are conjugated to three-armed DNA structures to achieve multivalent binding.

The peptides can serve as cost-effective replacement for monoclonal antibodies such as palivizumab—the so far only available prophylaxis against viral infections, e.g., RSV infections. The prophylaxis with the combination comprising the peptides or the nanostructure offers an attractive strategy for the prevention of RSV infection in high-risk populations.

The pharmaceutical combinations comprising peptides and/or nanostructures are useful for inhibiting entry of viruses, such as RSV, into mammalian cells. Further, the combinations comprising peptides and nanostructures are useful for inhibiting virus (e.g., RSV) spreading.

The present invention provides the following aspects and embodiments.
1. A compound, or a pharmaceutical composition comprising the compound, for use in a method for treating a viral infection caused by a virus belonging to a taxon selected from Duplodnaviria; Monodnaviria; Negarnaviricota; Pisuviricota; and Flavivirus;
wherein the compound has a structure according to Formula (I), an enantiomer, a diastereoisomer, a hydrate, solvate, a crystal form, a tautomer, or a pharmaceutically acceptable salt thereof:

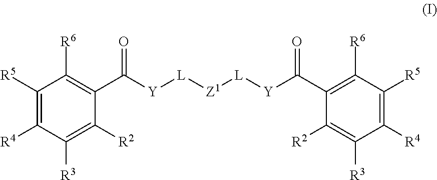

wherein:
L is, independently for each occurrence, optionally substituted C₁₋₆ alkylene, wherein carbon atoms from two different L groups can optionally form, together with Z¹, part of an optionally substituted 4- to 8-membered carbocyclic or heterocyclic ring;
Y is selected, independently for each occurrence, from O and NR⁷;
Z¹ is selected from bond, O, NR, CH₂, CH(R), CH(R¹), and CR(R¹);
R is selected from OH, optionally substituted C₁₋₆ alkyl, NR⁷R⁸, and

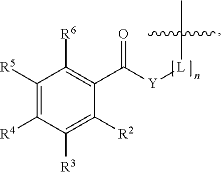

wherein n is 0 or 1;
R¹ is selected from OH, optionally substituted C₁₋₆ alkyl, and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, or R⁶ is OH, and/or wherein at least two occurrences of R², R³, R⁴, R⁵, or R⁶ form together part of an optionally substituted 4- to 8-membered heterocyclic ring containing O; and
R⁷ and R⁸ are selected, independently for each occurrence, from H and optionally substituted C₁₋₆ alkyl.
2. The compound or pharmaceutical composition for use according to aspect 1, wherein the virus belongs to a taxon selected from:
   (a) Heunggongvirae; Shotokuvirae; Negarnaviricota; Pisuviricota; and Flavivirus;
   (b) preferably selected from: Peploviricota; Cossaviricota; Haploviricotina; Pisuviricota; and Flavivirus;
   (c) more preferably selected from: Herviviricetes; Papovaviricetes; Monjiviricetes; Pisoniviricetes; and Flavivirus;
   (d) more preferably selected from Herpesvirales; Zurhausenvirales; Mononegavirales; Nidovirales, preferably Cornidovirineae; and Flavivirus;
   (e) more preferably selected from Herpesviridae, preferably Alphaherpesvirinae; Papillomaviridae, preferably Firstpapillomavirinae or Secondpapillomavirinae; Pneumoviridae; Coronaviridae, preferably Orthocoronavirinae; and Flavivirus;
   (f) most preferably selected from Simplexvirus; Alphapapillomavirus; Orthopneumovirus; Betacoronavirus, preferably Sarbecovirus; and Flavivirus.
3. The compound or pharmaceutical composition for use according to aspect 1 or 2, wherein
   L is, independently for each occurrence, C₁₋₆ alkylene, preferably linear or branched C₁, C₂, C₃, C₄, C₅ or C₅ alkylene, more preferably methylene, optionally substituted with one or more of halogen, OH, CN, NR⁷R⁸, C(O)NR⁷R⁸, C(O)OR⁷, and OR⁷, wherein carbon atoms from two L groups can optionally form, together with Z¹, part of a 4- to 8-membered carbocyclic or heterocyclic ring optionally substituted with one or more of halogen, OH, CN, NR⁷R⁸, C(O)NR⁷R⁸, C(O)OR⁷, and OR⁷;
   Y is selected, independently for each occurrence, from O and NR⁷;
   Z¹ is selected from bond, O, NR, CH₂, CH(R), CH(R¹), and CR(R¹);
   R is selected from: OH,
      linear or branched, acyclic or cyclic C₁₋₆ preferably C₁₋₃ alkyl, more preferably methyl, each of which can optionally substituted with one or more of halogen, OH, CN, NR⁷R⁸, C(O)NR⁷R⁸, C(O)OR⁷, and OR⁷,
      NR⁷R⁸, and

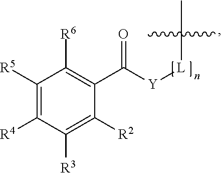

wherein n is 0 or 1, preferably 1;
R¹ is selected from: OH,
   linear or branched, acyclic or cyclic C₁₋₆ preferably C₁₋₃ alkyl, more preferably methyl, each of which can be optionally substituted with one or more of halogen, OH, CN, NR⁷R⁸, C(O)NR⁷R⁸, C(O)OR⁷, and OR⁷, and
   NR⁷R⁸;
R², R³, R⁴, R⁵, and R⁶ are selected, independently for each occurrence, from:
   H, halogen, OH, CN, NR⁷R⁸, C(O)NR⁷R⁸, and OR⁷,
   wherein at least one occurrence of R², R³, R⁴, R⁵, or R⁶ is OH, and/or wherein at least two occurrences of R², R³, R⁴, R⁵, and R⁶ form together part of a 4- to 8-membered heterocyclic ring containing O which can be optionally substituted with one or more of halogen, OH, CN, NR⁷R⁸, OR⁷, C(O)NR⁷R⁸, C(O)OR⁷, and OR⁷; and
R⁷ and R⁸ are selected, independently for each occurrence, from H, and linear or branched, acyclic or cyclic C₁₋₆ preferably C₁₋₃ alkyl, more preferably methyl, each of which can optionally substituted with one or more of halogen, OH, CN, NH₂, NH(C₁₋₆ alkyl), N(C₁₋₆ alkyl)₂C(O)NH₂, C(O)NH(C₁₋₆ alkyl), C(O)N(C₁₋₆ alkyl)₂, C(O)OH, C(O)O(C₁₋₆ alkyl), and O(C₁₋₆ alkyl), wherein each C₁₋₆ alkyl is independently selected and can be optionally substituted with one or more of halogen, OH, CN, NH₂, NH(C₁₋₆ alkyl), N(C₁₋₆ alkyl)₂C(O)NH₂, C(O)NH(C₁₋₆ alkyl), C(O)N(C₁₋₆ alkyl)₂, C(O)OH, C(O)O(C₁₋₆ alkyl), and O(C₁₋₆ alkyl).
4. The compound or pharmaceutical composition for use according to any one of the preceding aspects, wherein:
   Z¹ is selected from O, CH(R), CH(R¹), and CR(R¹).
5. The compound or pharmaceutical composition for use according to any one of the preceding aspects, wherein:
   Z¹ is selected from CH(R¹) and CR(R¹); and
   R¹ is selected from optionally substituted C₁₋₆ alkyl and NR⁷R⁸.
6. The compound or pharmaceutical composition for use according to any one of the preceding aspects, wherein:
   (a) at least one, preferably each occurrence of Y is NR⁷; and Z¹ is selected from CH(R¹) and CR(R¹); or
   (b) at least one occurrence, preferably each occurrence of Y is O; and Z¹ is selected from O, NR, CH₂, CH(R), and CH(R¹).
7. The compound or pharmaceutical composition for use according to any one of the preceding aspects, wherein:
   (a) on at least one of the phenyl rings in Formula (I), each of R³ and R⁴ is OH; and preferably each of R², R⁵, and R⁶ is H or OH; and/or
   (b) on at least one of the phenyl rings in Formula (I), each of R³ and R⁵ is OH; and preferably each of R², R⁴, and R⁶ is H or OH.
8. The compound or pharmaceutical composition for use according to any one of the preceding aspects, wherein on at least one, preferably on at least two, more preferably on three of the phenyl rings in Formula (I), three of R², R³, R⁴, R⁵, and R⁶ are OH.
9. The compound or pharmaceutical composition for use according to any one of the preceding aspects, wherein the compound of Formula (I) has a structure according to Formula (II):
   wherein:

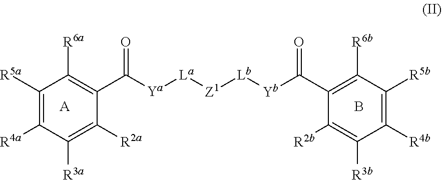

L^{a} is, independently for each occurrence, optionally substituted C₁₋₆ alkylene, preferably C₁₋₆ alkylene, preferably linear or branched C₁, C₂, C₃, C₄, C₅ or C₅ alkylene, more preferably methylene, wherein carbon atoms from two groups among L^{a}, L^{b}, and L^{c} can optionally form, together with Z¹, part of an optionally substituted 4- to 8-membered carbocyclic or heterocyclic ring;
L^{b} is, independently for each occurrence, optionally substituted C₁₋₆ alkylene, preferably C₁₋₆ alkylene, preferably linear or branched C₁, C₂, C₃, C₄, C₅ or C₅ alkylene, more preferably methylene, wherein carbon atoms from two groups among L^{a}, L^{b}, and L^{c} can optionally form, together with Z¹, part of an optionally substituted 4- to 8-membered carbocyclic or heterocyclic ring;
Y^{a} is selected, independently for each occurrence, from O and NR⁷;
Y^{b} is selected, independently for each occurrence, from O and NR⁷;
Z¹ is selected from bond, O, NR, CH₂, CH(R), CH(R¹), and CR(R¹);
R is selected from OH, optionally substituted C₁₋₆ alkyl, NR R⁸, and

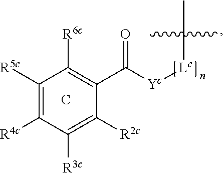

wherein n is 0 or 1; Y^{c} is selected, independently for each occurrence, from O and NR⁷; and L^{c} is, independently for each occurrence, optionally substituted C₁₋₆ alkylene, wherein carbon atoms from two groups among L^{a}, L^{b}, and L^{c} can optionally form, together with Z¹, part of an optionally substituted 4- to 8-membered carbocyclic or heterocyclic ring;
R¹ is selected from OH, optionally substituted C₁₋₆ alkyl, and NR⁷R⁸;
R^{2a}, R^{3a}, R^{4a}, R^{5a}, and R^{6a} are selected, independently for each occurrence, from H, halogen, OH, CN, NR⁷R⁸, C(O)NR⁷R⁸, and OR⁷,
R^{2b}, R^{3b}, R^{4b}, R^{5b}, and R^{6b} are selected, independently for each occurrence, from H, halogen, OH, CN, NR⁷R⁸, C(O)NR⁷R⁸, and OR⁷;
R^{2c}, R^{3c}, R^{4c}, R^{5c}, and R^{6c} are selected, independently for each occurrence, from H, halogen, OH, CN, NR⁷R⁸, C(O)NR⁷R⁸, and OR⁷;
wherein at least one occurrence of R^{2a}, R^{3a}, R^{4a}, R^{5a}, R^{6a}, R^{2b}, R^{3b}, R^{4b}, R^{5b}, R^{6b} R^{2c}, R^{3c}, R^{4c}, R^{5c}, or R^{6c} is OH, and/or wherein at least two occurrences of R^{2a}, R^{3a}, R^{4a}, R^{5a}, R^{6a}, R^{2b}, R^{3b}, R^{4b}, R^{5b}, R^{6b} R^{2c}, R^{3c}, R^{4c}, R^{5c}, or R^{6c} form together part of an optionally substituted 4- to 8-membered heterocyclic ring containing O; and
R⁷ and R⁸ are selected, independently for each occurrence, from H and optionally substituted C₁₋₆ alkyl.
10. The compound or pharmaceutical composition for use according to any one of the preceding aspects, wherein the compound of Formula (I) has a structure according to Formula (IIIa), (IIIb), (IIIc), or (IIId):

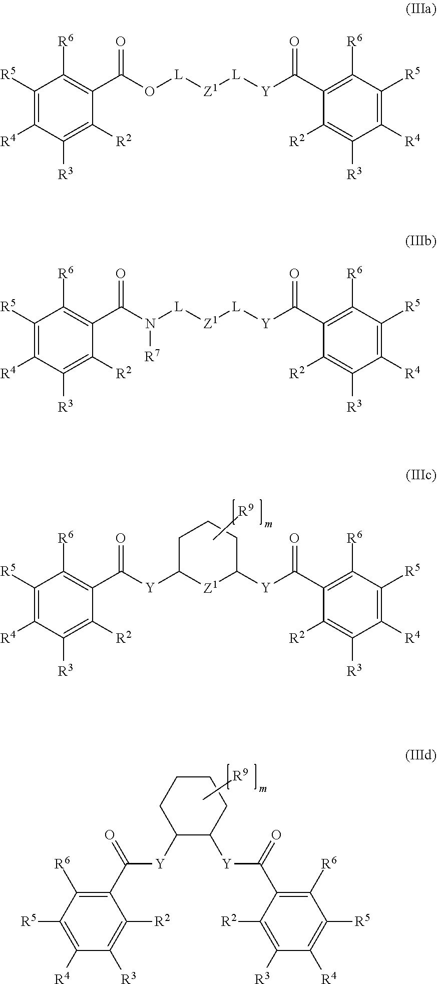

wherein:
R⁹ is independently selected from OH, CN, NR⁷R⁸, C(O)NR⁷R⁸, C(O)OR⁷, and OR⁷;
m is 0, 1, 2, 3, 4, 5, 6, 7, or 8; and
the remaining groups are defined as in any one of the preceding aspects.
11. The compound or pharmaceutical composition for use according to any one of the preceding aspects, wherein the compound of Formula (I) has a structure according to Formula (IVa), (IVb), (IVc), (IVd), (IVe), or (IVf):

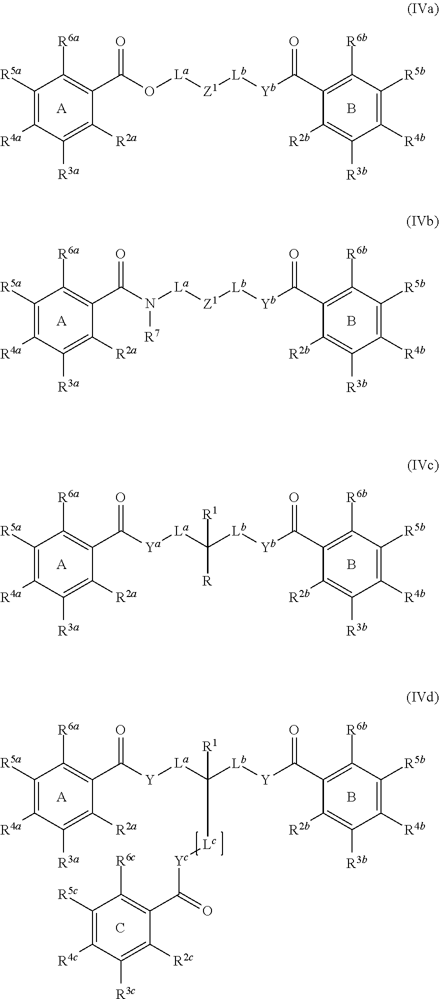

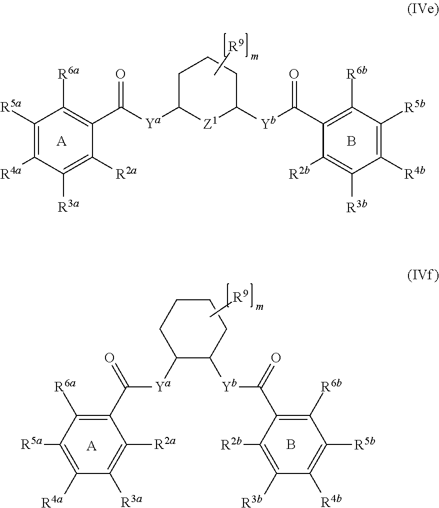

wherein all groups are defined as in any one of the preceding aspects.
12. A compound, or a pharmaceutical composition comprising the compound, for use in a method for treating a viral infection, wherein the compound has a structure as defined in any one of the preceding aspects, an enantiomer, a diastereoisomer, a hydrate, a solvate, a crystal form, a tautomer, or a pharmaceutically acceptable salt thereof; and wherein:
   L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
   Y is selected, independently for each occurrence, from O and NR⁷;
   Z¹ is selected from bond, O, CH(R), CH(R¹), and CR(R¹);
R is selected from OH, optionally substituted C₁₋₆ alkyl, NR⁷R⁸, and

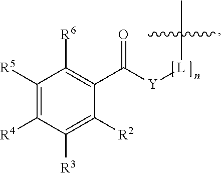

wherein n is 0 or 1;
R¹ is selected from OH, optionally substituted C₁₋₆ alkyl, and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, or R⁶ is OH, and/or wherein at least two occurrences of R², R³, R⁴, R⁵, or R⁶ form together part of an optionally substituted 4- to 8-membered heterocyclic ring containing O; and
R⁷ and R⁸ are selected, independently for each occurrence, from H and optionally substituted C₁₋₆ alkyl.
13. The compound or pharmaceutical composition for use according to aspect 12, wherein the viral infection is caused by a virus belonging to a taxon selected from:
   (a) Duplodnaviria; Monodnaviria; and Riboviria;
   (b) preferably selected from Heunggongvirae; Shotokuvirae; and Orthornavirae;
   (c) more preferably selected from Peploviricota; Cossaviricota; Negarnaviricota, preferably Haploviricotina; Pisuviricota; and Kitrinoviricota;
   (d) more preferably selected from Herviviricetes; Papovaviricetes; Monjiviricetes; Pisoniviricetes; and Flasuviricetes;
   (e) more preferably selected from Herpesvirales; Zurhausenvirales; Mononegavirales; Nidovirales, preferably Cornidovirineae; and Amarillovirales;
   (f) more preferably selected from Herpesviridae, preferably Alphaherpesvirinae; Papillomaviridae, preferably Firstpapillomavirinae or Secondpapillomavirinae; Pneumoviridae; Coronaviridae, preferably Orthocoronavirinae; and Flaviviridae;
   (g) most preferably selected from, preferably Simplexvirus; Alphapapillomavirus; Orthopneumovirus; Betacoronavirus, preferably Sarbecovirus; and Flavivirus.
14. The compound or pharmaceutical composition for use according to any one of the preceding aspects, wherein the virus is selected from Human alphaherpesvirus; Human papillomavirus; Respiratory syncytial virus; Severe acute respiratory syndrome-related coronavirus; and Zika virus.
15. The compound or pharmaceutical composition for use according to any one of the preceding aspects, wherein the virus is selected from HSV-1; HPV-16; RSV; SARS-CoV-2; and Zika virus.
16. The compound or pharmaceutical composition for use according to any one of the preceding aspects, wherein the virus is an enveloped virus.
17. A compound, or a pharmaceutical composition comprising the compound, wherein the compound has a structure as defined in any one of the preceding aspects, an enantiomer, a diastereoisomer, hydrate, a solvate, a crystal form, a tautomer, or a pharmaceutically acceptable salt thereof.
18. The compound or pharmaceutical composition according to aspect 17, wherein:
   L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
   Y is selected, independently for each occurrence, from O and NR⁷;
   Z¹ is selected from bond, CH(R), CH(R¹), and CR(R¹);
R is selected from OH, optionally substituted C₁₋₆ alkyl, NR⁷R⁸, and

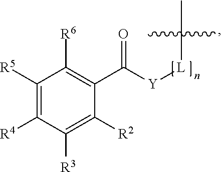

wherein n is 0 or 1;
R¹ is selected from optionally substituted C₁₋₆ alkyl and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence R², R³, R⁴, R⁵, and R⁶ is OH, and/or wherein at least two occurrences of R², R³, R⁴, R⁵, and R⁶ form together part of an optionally substituted 4- to 8-membered heterocyclic ring containing O; and
R⁷ and R⁸ are selected, independently for each occurrence, from H and optionally substituted C₁₋₆ alkyl.
19. The compound or pharmaceutical composition according to aspect 17 or 18, wherein if all occurrences of Y are O, then Z¹ is selected from bond, CH(R¹), and CR(R¹).
20. The compound or pharmaceutical composition according to any one of aspects 17 to 19, or the compound or pharmaceutical composition for use according to any one of aspects 1 to 16, wherein the compound does not have the structure

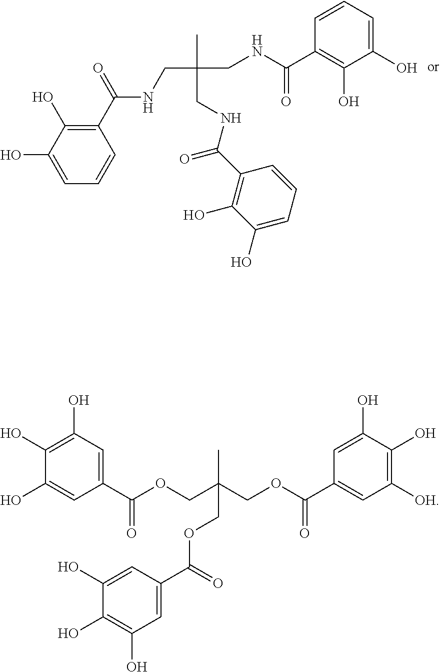 [SMILES: CC(CNC(=O)c1cccc(O)c1O)(CNC(=O)c1cccc(O)c1O)CNC(=O)c1cccc(O)c1O ; "or" ; CC(COC(=O)c1cc(O)c(O)c(O)c1)(COC(=O)c1cc(O)c(O)c(O)c1)COC(=O)c1cc(O)c(O)c(O)c1]

21. The compound or pharmaceutical composition according to any one of aspects 17 to 20, or the compound or pharmaceutical composition for use according to any one of aspects 1 to 16 or 20, wherein the compound does not have the structure

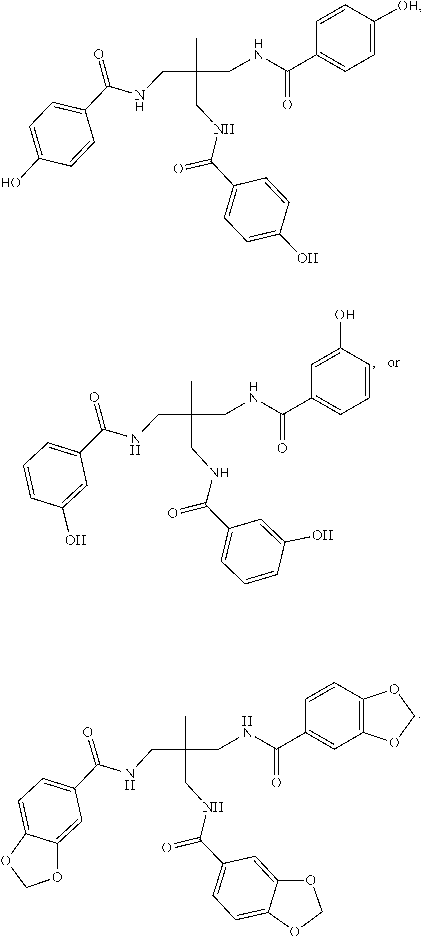 [SMILES: CC(CNC(=O)c1ccc(O)cc1)(CNC(=O)c1ccc(O)cc1)CNC(=O)c1ccc(O)cc1 ; CC(CNC(=O)c1cccc(O)c1)(CNC(=O)c1cccc(O)c1)CNC(=O)c1cccc(O)c1 ; CC(CNC(=O)c1ccc2c(c1)OCO2)(CNC(=O)c1ccc2c(c1)OCO2)CNC(=O)c1ccc2c(c1)OCO2]

22. A pharmaceutical combination comprising:
   a compound having a structure as defined in any one of the preceding aspects, an enantiomer, a diastereoisomer, hydrate, a solvate, a crystal form, a tautomer, or a pharmaceutically acceptable salt; and
   a peptide or a nanostructure;
   the peptide having:
      a length of 25 amino acids or less and comprising the sequence X₁—X₂—X₃—X₄—X₅—X₆—X₇—X₈—X₉—X₁₀—X₁₁—X₁₂— X₁₃ (SEQ ID NO: 1), wherein X₁ is Leu, Val or Ile, or a non-natural or natural analogue thereof, X₂ is Val, or a non-natural or natural analogue thereof, X₃ is Val, or a non-natural or natural analogue thereof, X₄ is a natural or non-natural amino acid, X₅ is a natural or non-natural amino acid, X₆ is a natural or non-natural amino acid, X₇ is Tyr, or a non-natural or natural analogue thereof, X₈ is Leu, Val, or Ile, or a non-natural or natural analogue thereof, X₉ is Pro, or a non-natural or natural analogue thereof, X₁₀ is a natural or non-natural amino acid, X₁₁ is a natural or non-natural amino acid, X₁₂ is a natural or non-natural amino acid, X₁₃ is Asp or Glu, or a non-natural or natural analogue thereof, and wherein at least one amino acid in the sequence pursuant to SEQ ID No: 1 is different from the amino acid in the sequence LVVSTTYLPHYFD (SEQ ID No: 3) at the corresponding position, or a peptidomimetic or retro-inverso peptide thereof; or
      a length of 25 amino acids or less and comprising the sequence X₁—X₂—X₃—X₄—X₅—X₆—X₇—X₈—X₉—X₁₀—X₁₁—X₁₂— X₁₃ (SEQ ID NO: 2), wherein X₁ is Leu, Val or Ile, or a non-natural or natural analogue thereof, X₂ is Val, or a non-natural or natural analogue thereof, X₃ is Val, or a non-natural or natural analogue thereof, X₄ is a natural or non-natural amino acid, X₅ is a natural or non-natural amino acid, X₆ is a natural or non-natural amino acid, X₇ is Tyr, or a non-natural or natural analogue thereof, X₈ is Leu, Val, or Ile, or a non-natural or natural analogue thereof, X₉ is Pro, or a non-natural or natural analogue thereof, X₁₀ is a natural or non-natural amino acid, X₁₁ is a natural or non-natural amino acid, X₁₂ is a natural or non-natural amino acid, X₁₃ is Asp or Glu, or a non-natural or natural analogue thereof, X₁₄ is a natural or non-natural amino acid, and wherein at least one amino acid in the sequence pursuant to SEQ ID No: 2 is different from the amino acid in the sequence LVVSTTYLPHYFDN (SEQ ID No: 5) at the corresponding position,
or a peptidomimetic or retro-inverso peptide thereof;
the nanostructure comprising:
   a) a nucleic acid scaffold; and
   b) at least two peptide moieties, wherein the sequence of each of the at least two peptide moieties is independently selected from the sequence of a peptide as specified above, wherein the nucleic acid scaffold is selected from the group of:
   i) a linear nucleic acid scaffold, wherein the at least two peptide moieties are each attached at or near different ends of the nucleic acid scaffold; and
   ii) a branched nucleic acid scaffold, wherein the at least two peptide moieties are each attached to a different branch of the scaffold.
23. The combination of aspect 22, wherein the peptide has a length of 20 amino acids or less, or a peptidomimetic or retro-inverso peptide thereof.
24. The combination of aspects 22 or 23, wherein in the peptide, 2 or more amino acids in the sequence pursuant to SEQ ID No: 1 are different from the amino acid in the sequence LVVSTTYLPHYFD (SEQ ID No: 3) at the corresponding position, or wherein 2 or more amino acids in the sequence pursuant to SEQ ID No: 2 are different from the amino acid in the sequence LVVSTTYLPHYFDN (SEQ ID No: 5) at the corresponding position, or a peptidomimetic or retro-inverso peptide thereof.
25. The combination of any of aspects 22 to 24, wherein at least one amino acid in the sequence of SEQ ID No: 1 or 2 is replaced by a non-natural or natural analogue thereof in the peptide, or a peptidomimetic or retro-inverso peptide thereof.
26. The combination of any of aspects 22 to 25, wherein at least one amino acid in the sequence of SEQ ID No: 1 or 2 is replaced by a non-natural or natural analogue thereof in the peptide at one or more positions selected from X₁, X₂, X₃, X₇, X₈, X₉ and X₁3.
27. The combination according to any of aspects 22 to 26, wherein the peptide, or a peptidomimetic or retro-inverso peptide thereof:
   comprises L-amino acids, D-amino acids, or a mixture thereof, comprises at least one backbone-modified amino acid,
   is a cyclic molecule,
   comprises at least one non-peptide moiety, preferably selected from a coupling group, a PEG moiety, a detectable label, a protective group, a lipid moiety and a sugar moiety, or comprises one of more post-translational modifications, and/or
   is covalently or non-covalently bound to a scaffold.
28. The combination of any of aspects 22 to 27, wherein the peptide comprises or consists of a sequence selected from:

(SEQ ID No: 6)
VVVSTTYLPHYFDN
(SEQ ID No: 7)
IVVSTTYLPHYFDN
(SEQ ID No: 8)
[2-Abu]VVSTTYLPHYFDN
(SEQ ID No: 9)
LV[Chg]STTYLPHYFDN
(SEQ ID No: 10)
LVVSTT[Tyr3-I]LPHYFDN,
(SEQ ID No: 11)
[Chg][Cpa]VSTT[Tyr3,5-12]LPHYFDN,
and
(SEQ ID No: 12)
[Chg][Nle][Chg]STTYLPHYFDN,

or a peptidomimetic or a retro-inverso peptide thereof.
29. The combination of any of aspects 22 to 28, wherein:
   the nucleic acids of the nucleic acid scaffold are double stranded over a sufficient length to remain stable in physiological condition, each of the nucleic acid strands of the scaffold is made of a nucleic acid selected from the group comprising DNA and chemically stabilized variants thereof, RNA and chemically stabilized variants thereof, locked nucleic acid (LNA), peptide nucleic acid (PNA), and xeno-nucleic acids (XNA), the peptide moieties each bind to RSV Fusion (F) glycoprotein with a KD 10 μM or less under physiological conditions,
   the peptide moieties comprise or consist of a sequence of any of SEQ ID NOs: 1 to 16, or a retro-inverso peptide thereof,
   the nucleic acid scaffold is branched and at least three peptide moieties are each attached to a different branch of the nucleic acid scaffold, preferably wherein all the branches of the scaffold emanate from a single junction,
   the nucleic acid scaffold is branched and at least three peptide moieties are each attached to a different branch of the nucleic acid scaffold, wherein all the branches of the scaffold emanate from a single junction, and wherein each branch of the nucleic acid scaffold is about 10 to 25 nucleotides in length, and/or
   the nucleic acid scaffold is branched and three peptide moieties are each attached to a different branch of the nucleic acid scaffold via the N-terminus of the peptide moieties, the C-terminus of the peptide moieties, or via an amino acid side chain, wherein the nanostructure optionally further comprises a detectable label.
30. A pharmaceutical composition comprising the combination of any of aspects 22 to 29, and optionally one or more pharmaceutically acceptable adjuvants and/or excipients.
31. A nucleic acid comprising a sequence encoding a peptide as defined in of any of aspects 22 to 29.
32. A combination according to any of aspects 22 to 29, or a pharmaceutical composition of aspect
30, or a nucleic acid of aspect 31 in combination with a compound having a structure as defined in any one of aspects 1 to 21, an enantiomer, a diastereoisomer, hydrate, a solvate, a crystal form, a tautomer, or a pharmaceutically acceptable salt, for use as a medicament.
33. A combination according to any of aspects 22 to 29, or a pharmaceutical composition of aspect 30, or a nucleic acid of aspect 31 in combination with a compound having a structure as defined in any one of aspects 1 to 21, an enantiomer, a diastereoisomer, hydrate, a solvate, a crystal form, a tautomer, or a pharmaceutically acceptable salt, for use in the treatment, prophylactic treatment, or amelioration of a viral infection; preferably a viral infection as defined in any of aspects 1, 2, 13, 14, 15 and 16; more preferably a Respiratory Syncytial Virus (RSV) infection.
34. Use of combination according to any of aspects 22 to 29, or a pharmaceutical composition of aspect 30, or a nucleic acid of aspect 31 in combination with a compound having a structure as defined in any one of aspects 1 to 21, an enantiomer, a diastereoisomer, hydrate, a solvate, a crystal form, a tautomer, or a pharmaceutically acceptable salt, as an in vitro RSV antiviral agent; or as an in vitro diagnostic agent for detecting RSV.
35. A combination according to any of aspects 22 to 29, or a pharmaceutical composition of aspect 30, or a nucleic acid of aspect 31 in combination with a compound having a structure as defined in any one of aspects 1 to 21, an enantiomer, a diastereoisomer, hydrate, a solvate, a crystal form, a tautomer, or a pharmaceutically acceptable salt, for use in the in the diagnosis of a viral infection; preferably a viral infection as defined in any of aspects 1, 2, 13, 14, 15 and 16; more preferably a Respiratory Syncytial Virus (RSV) infection.

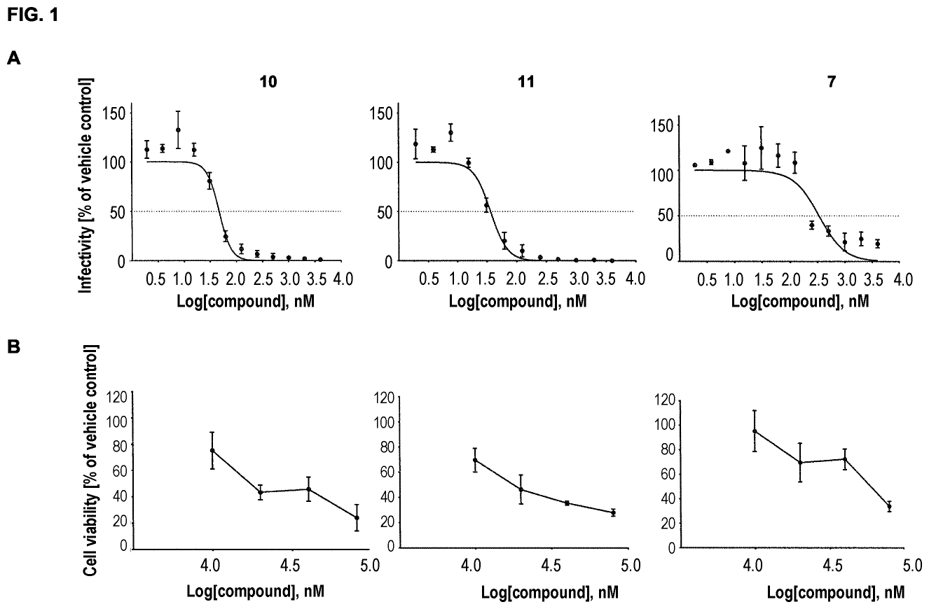

In the context of the pharmaceutical combination to which above aspects 22 to 35 and appended claim 15 relate, protection is or may be sought for any features disclosed on pages 1 to 66 and FIGS. 1 to 12 of WO 2020/212576 A1, the entire disclosure of which, including all definitions, features and examples, explicitly and clearly belongs to the description of the present invention, as a part of said pharmaceutical combination.

The expression “alkyl” as used herein, unless specifically limited, denotes a suitable alkyl group, e.g., C₁₋₆ alkyl group, e.g. C₁₋₄ alkyl group. Alkyl groups may be straight chain or branched. Suitable alkyl groups include, for example, methyl, ethyl, propyl (e.g. n-propyl and isopropyl), butyl (e.g. n-butyl, iso-butyl, sec-butyl and tert-butyl), pentyl (e.g. n-pentyl), hexyl (e.g. n-hexyl). The alkyl groups may be linear or branched, acyclic or cyclic C₁₋₆ preferably C₁₋₃ alkyl, more preferably methyl.

The term “alkyl” also comprises cycloalkyl groups. The expression “cycloalkyl”, denotes a suitable alicyclic alkyl group, e.g., C₃₋₆ cycloalkyl group. Exemplary cycloalkyl groups include cyclopropyl, cyclobutyl, cyclopentyl, cyclohexyl. A most suitable number of ring carbon atoms is three to six.

The expressions “carbocyclyl” and “carbocyclic”, unless specifically limited, denote any ring system in which all the ring atoms are carbon and which contains between three and twelve ring carbon atoms, suitably between three and ten carbon atoms and more suitably between three and eight carbon atoms. Carbocyclyl groups may be saturated or partially unsaturated, but do not include aromatic rings. Examples of carbocyclyl groups include monocyclic, bicyclic, and tricyclic ring systems, in particular monocyclic and bicyclic ring systems. Other carbocyclyl groups include bridged ring systems (e.g. bicyclo[2.2.1]heptenyl). A specific example of a carbocyclyl group is a cycloalkyl group. A further example of a carbocyclyl group is a cycloalkenyl group.

The expression “aryl”, unless specifically limited, denotes a C₆₋₁₂ aryl group, suitably a C₆₋₁₀ aryl group, more suitably a C₆₋₈ aryl group. Aryl groups will contain at least one aromatic ring (e.g. one, two or three rings). An example of a typical aryl group with one aromatic ring is phenyl. An example of a typical aryl group with two aromatic rings is naphthyl.

The expressions “heterocyclyl” and “heterocyclic”, unless specifically limited, refer to a carbocyclyl group wherein one or more (e.g. 1, 2 or 3) ring atoms are replaced by heteroatoms selected from N, S and O. A specific example of a heterocyclyl group is a cycloalkyl group (e.g. cyclopentyl or more particularly cyclohexyl) wherein one or more (e.g. 1, 2 or 3, particularly 1 or 2, especially 1) ring atoms are replaced by heteroatoms selected from N, S or O. Exemplary heterocyclyl groups containing one hetero atom include pyrrolidine, tetrahydrofuran and piperidine, and exemplary heterocyclyl groups containing two hetero atoms include morpholine and piperazine. A further specific example of a heterocyclyl group is a cycloalkenyl group (e.g. a cyclohexenyl group) wherein one or more (e.g. 1, 2 or 3, particularly 1 or 2, especially 1) ring atoms are replaced by heteroatoms selected from N, S and O. An example of such a group is dihydropyranyl (e.g. 3,4-dihydro-2H-pyran-2-yl-).

The expression “heteroaryl”, unless specifically limited, denotes an aryl residue, wherein one or more (e.g. 1, 2, 3, or 4, suitably 1, 2 or 3) ring atoms are replaced by heteroatoms selected from N, S and O, or else a 5-membered aromatic ring containing one or more (e.g. 1, 2, 3, or 4, suitably 1, 2 or 3) ring atoms selected from N, S and O. Heteroaryl groups represent a particular subtype within the general class of “heterocyclyl” or “heterocyclic” groups. Exemplary monocyclic heteroaryl groups having one heteroatom include: five membered rings (e.g. pyrrole, furan, thiophene); and six membered rings (e.g. pyridine, such as pyridin-2-yl, pyridin-3-yl and pyridin-4-yl). Exemplary monocyclic heteroaryl groups having two heteroatoms include: five membered rings (e.g. pyrazole, oxazole, isoxazole, thiazole, isothiazole, imidazole, such as imidazol-1-yl, imidazol-2-yl imidazol-4-yl); six membered rings (e.g. pyridazine, pyrimidine, pyrazine). Exemplary monocyclic heteroaryl groups having three heteroatoms include: 1,2,3-triazole and 1,2,4-triazole. Exemplary monocyclic heteroaryl groups having four heteroatoms include tetrazole. Exemplary bicyclic heteroaryl groups include: indole (e.g. indol-6-yl), benzofuran, benzothiophene, quinoline, isoquinoline, indazole, benzimidazole, benzothiazole, quinazoline and purine.

The expression “alk”, for example in the expression “alkylene” may be interpreted in accordance with the definition of “alkyl”. Exemplary alkoxy groups include methoxy, ethoxy, propoxy (e.g. n-propoxy), butoxy (e.g. n-butoxy), pentoxy (e.g. n-pentoxy), and hexoxy (e.g. n-hexoxy). Exemplary haloalkyl groups include fluoroalkyl e.g. CF₃; exemplary haloalkoxy groups include fluoroalkyl e.g. OCF₃.

The term “halogen” or “halo” comprises fluorine (F), chlorine (Cl), bromine (Br) and iodine (I).

The terms “hydrogen” or “H” as used herein encompass all isotopes of hydrogen, in particular protium (¹H) and deuterium (²H, also denoted as D).

The term “optionally substituted” refers to optional substitution by one or several substituents independently selected from: halogen, OH, CN, NH₂, NH(C₁₋₆ alkyl), N(C₁₋₆ alkyl)₂C(O)NH₂, C(O)NH(C₁₋₆ alkyl), C(O)N(C₁₋₆ alkyl)₂, C(O)OH, C(O)O(C₁₋₆ alkyl), and O(C₁₋₆ alkyl), wherein each C₁₋₆ alkyl is independently selected and can be optionally substituted with one or more of halogen, OH, CN, NH₂, NH(C₁₋₆ alkyl), N(C₁₋₆ alkyl)₂C(O)NH₂, C(O)NH(C₁₋₆ alkyl), C(O)N(C₁₋₆ alkyl)₂, C(O)OH, C(O)O(C₁₋₆ alkyl), and O(C₁₋₆ alkyl). Further possible substituents encompassed by the meaning of “optionally substituted” may encompass C₁₋₆ alkyl, C₁₋₆ heteroalkyl, C₁₋₆ carbocyclyl, C₁₋₅ heterocyclyl and C₁₋₅ heteroaryl group, each of which may be substituted by one or several halogen atoms and/or hydroxyl groups; a halogen atom; a cyano group; a primary, secondary or tertiary amino group; a hydroxyl group; and a carboxyl group. Each of the preceding substituents may be further optionally substituted; preferably with one or more of the substituents selected from the preceding lists; and/or with further substituents comprising a total of no more than 10 non-H atoms.

In any of the embodiments, aspects or claims, or the compound preferably does not have the structure of one or more, more preferably any of the compounds listed below:

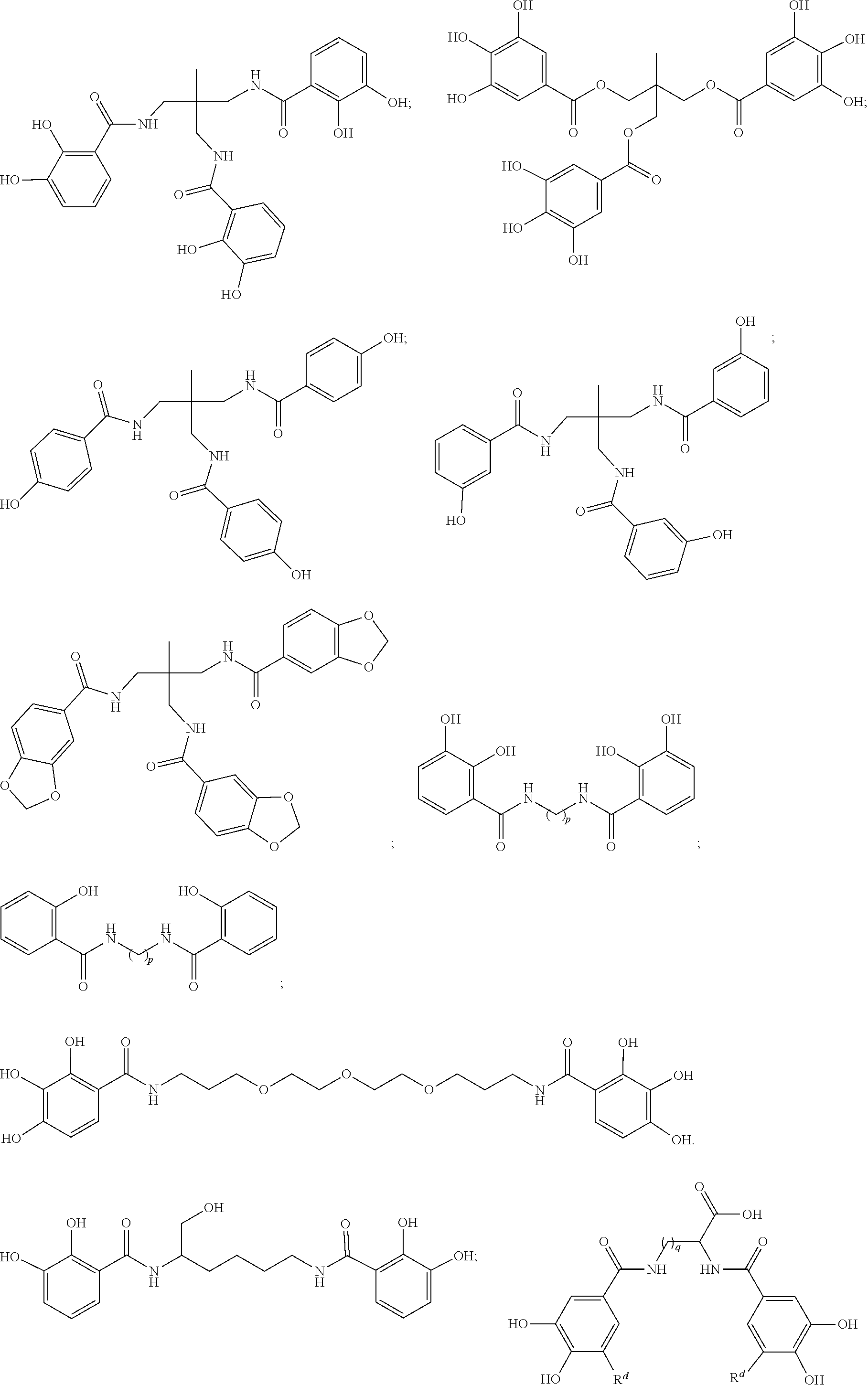

wherein p is 2, 4 or 12, preferably 2-12; q is 1, 2, 3 or 4, preferably 1-4, and R^{d} is H or OH;

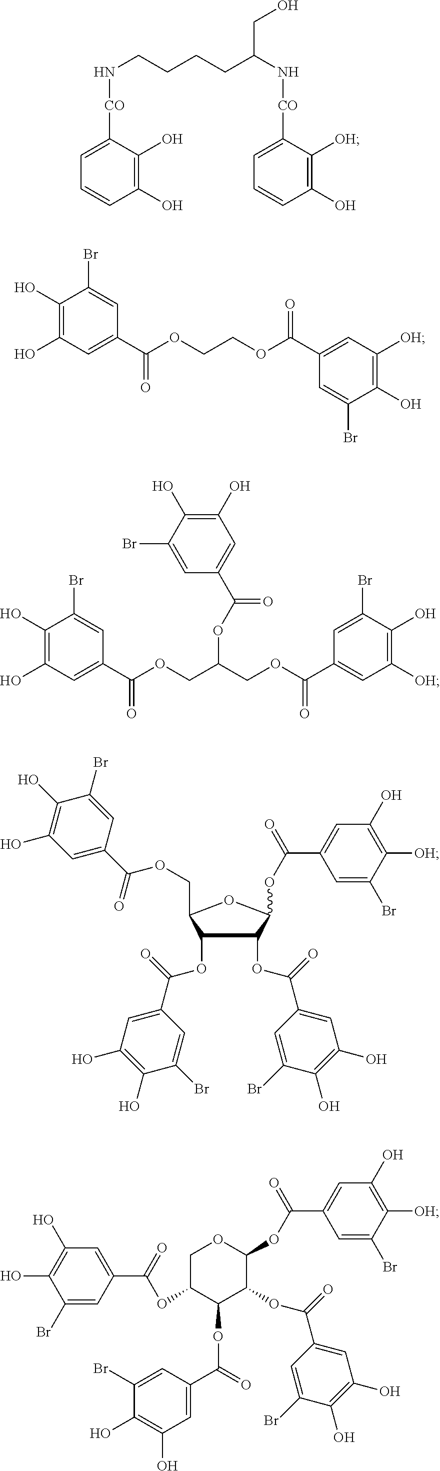

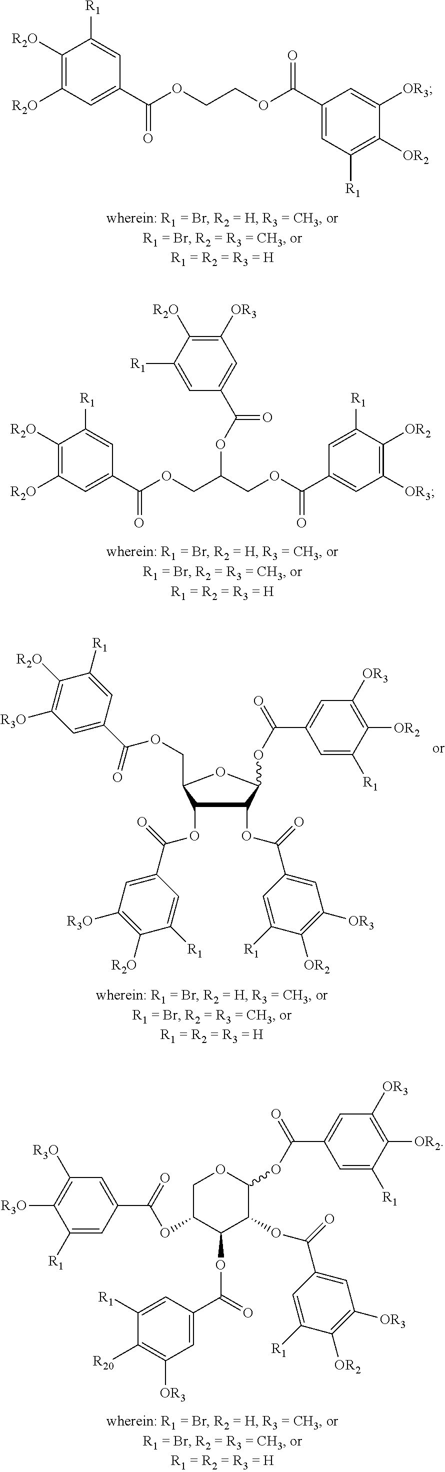

### Stereoisomers

All possible stereoisomers of the claimed compounds are included in the present invention.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base, or by salt formation with an optically active base, such as quinine, quinidine, quinotoxine, cinkotoxine, (S)-phenylethylamine, (1R,2S)-ephedrine, (R)-phenylglycinol, (S)-2-aminobutanol, followed by fractional crystallization and regeneration of the free acid. The compounds may also be resolved by formation of diastereomeric esters or amides, followed by chromatographic separation and removal of the chiral auxiliary. Alternatively, the compounds may be resolved using a chiral HPLC column.

### Pharmaceutically Acceptable Salts

Salts, hydrates and solvates of the compounds of Formula I are suitable for use in medicine are those wherein the counter-ion or associated solvent is pharmaceutically acceptable. However, salts, hydrates and solvates having non-pharmaceutically acceptable counter-ions or associated solvents are within the scope of the present invention, for example, for use as intermediates in the preparation of other compounds and their pharmaceutically acceptable salts, hydrates and solvates.

Suitable salts according to the invention include those formed with both organic and inorganic acids or bases. Pharmaceutically acceptable acid addition salts include those formed from hydrochloric, hydrobromic, sulfuric, nitric, citric, tartaric, phosphoric, lactic, pyruvic, acetic, trifluoroacetic, triphenylacetic, sulfamic, sulfanilic, succinic, oxalic, fumaric, maleic, malic, mandelic, glutamic, aspartic, oxaloacetic, methanesulfonic, ethanesulfonic, arylsulfonic (for example p-toluenesulfonic, benzenesulfonic, naphthalenesulfonic or naphthalenedisulfonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalenes-acrylic), benzoic, 4-methoxybenzoic, 2- or 4-hydroxybenzoic, 4-chlorobenzoic, 4-phenylbenzoic, benzeneacrylic (for example 1,4-benzenediacrylic), isethionic acids, perchloric, propionic, glycolic, hydroxyethanesulfonic, pamoic, cyclohexanesulfamic, salicylic, saccharinic and trifluoroacetic acid.

Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

All pharmaceutically acceptable acid addition salt forms of the compounds of the present invention are intended to be embraced by the scope of this invention.

The peptides or nanostructures contained in the combinations, or the pharmaceutical compositions described herein, can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

### Polymorph Crystal Forms, Solvates, Hydrates

Furthermore, some of the individual crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention. The compounds, including their salts, can also be obtained in the form of their hydrates, or include other solvents used for their crystallization. In view of the close relationship between the free compounds and the compounds in the form of their salts, hydrates or solvates, whenever a compound is referred to in this context, a corresponding salt, solvate or polymorph is also intended, provided such is possible or appropriate under the circumstances.

### Tautomers

As used herein, the term “tautomer” refers to the migration of protons between adjacent single and double bonds. The tautomerization process is reversible. Compounds described herein can undergo any possible tautomerization that is within the physical characteristics of the compound.

### Pharmaceutical Compositions and Dosage Forms

The pharmaceutical composition according to the present invention comprises a compound as described above, and a pharmaceutically acceptable excipient.

As used herein, the term “pharmaceutical composition” is intended to encompass a product comprising the claimed compounds in the therapeutically effective amounts, as well as any product that results, directly or indirectly, from combinations of the claimed compounds. As used herein, the term “excipient” refers to a carrier, a binder, a disintegrator and/or a further suitable additive for galenic formulations, for instance, for liquid oral preparations, such as suspensions, elixirs and solutions; and/or for solid oral preparations, such as, for example, powders, capsules, gelcaps and tablets. Carriers, which can be added to the mixture, include necessary and inert pharmaceutical excipients, including, but not limited to, suitable suspending agents, lubricants, flavorants, sweeteners, preservatives, coatings, granulating agents, dyes, and coloring agents.

Suitable the pharmaceutical dosage forms include but are not limited to oral, ophthalmic, inhalation, parenteral, topical, suppository etc. Preferably, the dosage forms can be powders, capsules, gelcaps and tablets, pills, syrups, pastes, ophthalmic solutions, eye drops, eye lotions, ophthalmic suspensions, ophthalmic ointments, ophthalmic emulsions, aerosols, inhalers, nebulizers, vaporizers, creams, gels, ointments, balms, lotions, ear drops, skin patches, suppositories, etc.

The compounds, pharmaceutical compositions or pharmaceutical combinations described herein described herein may be administered in any manner including, but not limited to, orally, parenterally, sublingually, transdermally, transmucosally, topically, via inhalation, via buccal or intranasal administration, or combinations thereof. Parenteral administration includes, but is not limited to, intravenous, intraarterial, intra-peritoneal, subcutaneous and intramuscular. In a preferred embodiment, the composition or combination is formulated, in accordance with routine procedures, as a pharmaceutical composition adapted for intranasal, or intravenous administration to human beings.

Typically, compositions or combinations for intravenous administration are solutions in sterile isotonic aqueous buffer. Where necessary, the composition or combination may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition or combination is to be administered by infusion, it can be dispensed with an infusion bottle containing sterile pharmaceutical grade water or saline. Where the composition or combination is administered by injection, an ampoule of sterile water or saline for injection can be provided so that the ingredients may be mixed prior to administration.

Preferably, the pharmaceutical composition or combination described herein is formulated for intranasal administration. The compounds, pharmaceutical compositions or combinations can be formulated, for example, in liquid form as nose drops, spray, or suitable for inhalation, as powder, as cream, or as emulsion. For straightforward application, the compound, pharmaceutical composition or combination is preferably supplied in a vessel appropriate for distribution of the peptide or nanostructures in the form of nose drops or an aerosol. More preferably, compounds, pharmaceutical compositions or pharmaceutical combinations described herein are administered by inhalation or spraying.

Preferably, the compounds, pharmaceutical compositions or pharmaceutical combinations described herein are formulated for a method of administration to the lungs of a subject by inhalation of an aerosol by the subject. Typically, the aerosol is delivered to the endobronchial space of airways from the subject. For example, the compounds, pharmaceutical compositions or pharmaceutical combinations may be delivered by a dry powder inhaler or by a metered dose inhaler.

### Therapeutic Applications

The present disclosure provides compounds and/or a pharmaceutical compositions as described above for use in a method for therapy or prophylaxis of viral infections. The present disclosure also provides a method for therapy or prophylaxis of viral infections the method comprising administering a therapeutically effective amount of said compound or composition to a subject in need thereof.

The term “subject” as used herein refers to an animal, preferably a mammal, most preferably a human, who is or has been the object of treatment, therapy, prophylaxis, observation or experiment.

The term “therapeutically effective amount” as used herein means that amount of active compound or pharmaceutical agent that elicits the biological or medicinal response in a tissue system, animal or human being sought by a researcher, veterinarian, medical doctor or other clinician, which includes alleviation of the symptoms of the disease or disorder being treated.

In the context of the present invention, the term “treatment” is intended to encompass any kind of therapeutic or prophylactic treatment of the human or animal body. The terms “therapy” and “therapeutic treatment” cover prophylactic methods of treating a disease, curative methods of treating a disease, and/or methods for alleviation of symptoms of a disease. The terms “prophylactic treatment”, “preventive treatment” and “preventing” have the same meaning and denote a treatment aiming at maintaining health by preventing ill effects that would otherwise arise.

Viral infection which can preferably be treated with the compounds disclosed herein are those caused by specific viruses or viruses belonging to taxa shown in Table u below. The classification in Table 1 is according to the ICTV Virus Taxonomy: 2020 Release. Human papillomaviruses (HPV), in particular Alpha papillomaviruses (alpha-PVs) and the classification of their viral variants are described in more detail in Burk R D, et al. Classification and nomenclature system for human Alphapapillomavirus variants: general features, nucleotide landmarks and assignment of HPV6 and HPV11 isolates to variant lineages. Acta Dermatovenerol Alp Pannonica Adriat. 2011 September; 20(3):113-23. PMID: 22131111; PMCID: PMC3690374.

TABLE 1
Preferred viruses and their taxonomic classification

| | | | | | |
|---|---|---|---|---|---|
| Realm | Duplodnaviria | Monodnaviria | Riboviria | | |
| Kingdom | Heunggongvirae | Shotokuvirae | Orthornavirae | | |
| Phylum | Peploviricota | Cossaviricota | Negarnaviricota | Pisuviricota | Kitrinoviricota |
| Subphylum | | | Haploviricotina | | |
| Class | Herviviricetes | Papovaviricetes | Monjiviricetes | Pisoniviricetes | Flasuviricetes |
| Order | Herpesvirales | Zurhausenvirales | Mononegavirales | Nidovirales | Amarillovirales |
| Suborder | | | | Cornidovirineae | |
| Family | Herpesviridae | Papillomaviridae | Pneumoviridae | Coronaviridae | Flaviviridae |
| Subfamily | Alphaherpesvirinae | Firstpapillomavirinae | | Orthocoronavirinae | |
| Genus | Simplexvirus | Alphapapillomavirus, | Orthopneumovirus | Betacoronavirus | Flavivirus |
| | | Betapapillomavirus, or | | | |
| | | Gammapapillomavirus | | | |
| Subgenus | | | | Sarbecovirus | |
| Species | Human | Alphapapillomavirus 9 | Human | Severe acute | Zika virus |
| | alphaherpesvirus | | orthopneumovirus | respiratory | (ZIKV) |
| | 1 | | (RSV) | syndrome-related | |
| | (HSV-1) | | | coronavirus | |
| Subspecies, | | HPV-16 | | SARS-CoV-2 | |
| Genotype or | | | | | |
| Variant | | | | | |
| Genome | dsDNA | ssDNA | ssRNA(−) | ssRNA(+) | ssRNA(+) |
| Composition | | | | | |

### EXAMPLES

### Example 1: Synthesis of Small Molecule Compounds

### Synthesis of Ester Derivatives

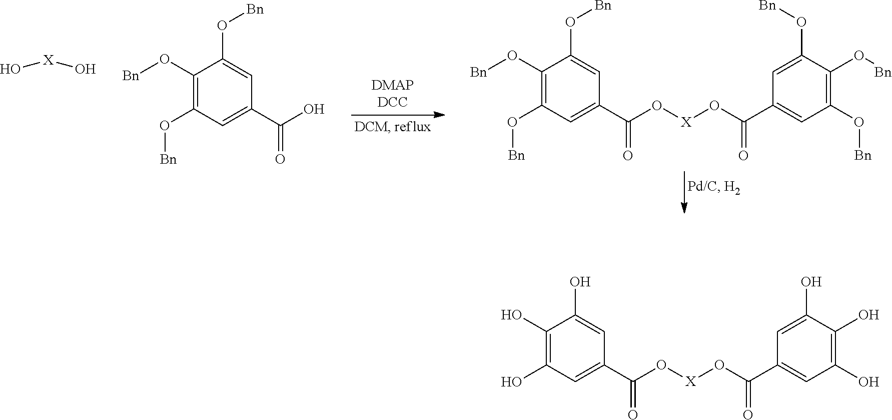

### Step 1

A solution of the respective diol (1 eq) was dissolved in DCM (0.1 M). 3,4,5-Tris(benzyloxy)benzoic acid (2.1 eq), DCC (2.3 eq) and DMAP (2.2 eq) were added and the mixture was heated to reflux for 18 hours. After cooling the formed precipitate was filtered off. The filtrate was evaporated and the residue was purified by flash chromatography (silica, chloroform).

### Step 2

The ester obtained in step 1 was dissolved in MeOH, treated with a catalytic amount of Pd/C and hydrogenated at 1.5 bar overnight. The mixture was filtered and the volatiles were evaporated. The crude was purified by semi-preparative HPLC.

For the synthesis of the triesters, 2 eq of 3,4,5-Tris(benzyloxy)benzoic acid, 3.3 eq of DMAP and 3.4 eq of DCC were used in the first step, and chloroform was used as the solvent in the second step.

### Compounds

### Compound 1: 3-(3,4,5-trihydroxybenzoyl)oxypropyl 3,4,5-trihydroxybenzoate

The compound was synthesized from 1,3-propandiol (2 mmol), 3,4,5-tris(benzyloxy)benzoic acid (4.2 mmol), DCC (4.6 mmol) and DMAP (4.4 mmol) as described above. Yield: 15% (115 mg, 0.3 mmol), ¹H-NMR, DMSO, 400 MHz: δ 2.08 (quin, 2H, ³J=6.4 Hz), 4.29 (t, 4H, ³J=6.4 Hz), 6.96 (s, 4H), 8.94 (br s, 2H), 9.26 (br s, 4H), HPLC: 8.96 min (98%), APCI-MS: m/z 381.1 [M+H]⁺

### Compound 2: 5-(3,4,5-trihydroxybenzoyl)oxypentyl 3,4,5-trihydroxybenzoate

The compound was synthesized from 1,3-pentanediol (2 mmol), 3,4,5-tris(benzyloxy)benzoic acid (4.2 mmol), DCC (4.6 mmol) and DMAP (4.4 mmol) as described above. Yield: 5% (42 mg, 0.1 mmol), ¹H-NMR, DMSO, 400 MHz: δ 1.45-1.53 (m, 2H), 1.70-1.77 (m, 4H), 4.17-4.21 (m, 4H), 6.96 (s, 4H), 8.92 (s, 2H), 9.25 (s, 4H), HPLC: 10.93 min (100%), ESI-MS: m/z 407.2 [M−H]⁻

### Compound 3: 8-(3,4,5-trihydroxybenzoyl)oxyoctyl 3,4,5-trihydroxybenzoate

The compound was synthesized from 1,3-octanediol (2 mmol), 3,4,5-tris(benzyloxy)benzoic acid (4.2 mmol), DCC (4.6 mmol) and DMAP (4.4 mmol) as described above. Yield: 11% (95 mg, 0.21 mmol), ¹H-NMR, DMSO, 400 MHz: δ 1.36-1.40 (br s, 8H), 1.63-1.70 (m, 4H), 4.14-4.18 (m, 4H), 6.95 (s, 4H), 8.92 (br s, 2H), 9.25 (br s, 4H), HPLC: 13.6 min (100%), ESI-MS: m/z 449.2 [M−H]⁻

### Compound 4: [2,2-dimethyl-3-(3,4,5-trihydroxybenzoyl)oxy-propyl]3,4,5-trihydroxybenzoate

The compound was synthesized from 2,2-dimethyl-1,3-propanediol (2 mmol), 3,4,5-tris(benzyloxy)benzoic acid (4.2 mmol), DCC (4.6 mmol) and DMAP (4.4 mmol) as described above. Yield: 20% (158 mg, 0.39 mmol), ¹H-NMR, DMSO, 400 MHz: δ 1.08 (s, 6H), 4.06 (s, 4H), 6.98 (s, 4H), 8.80-9.40 (m, 6H), HPLC: 11.09 min (100%), ESI-MS: m/z 409.2 [M+H]⁺

### Compound 5: 2-[2-(3,4,5-trihydroxybenzoyl)oxyethoxy]ethyl 3,4,5-trihydroxybenzoate

The compound was synthesized from 2-(2-hydroxyethoxy)ethanol (2 mmol), 3,4,5-tris(benzyloxy)benzoic acid (4.2 mmol), DCC (4.6 mmol) and DMAP (4.4 mmol) as described above. Yield: 20% (158 mg, 0.39 mmol), ¹H-NMR, DMSO, 400 MHz: δ 3.76-3.78 (m, 4H), 4.29-4.32 (m, 4H), 6.97 (s, 4H), 8.95 (br s, 2H), 9.27 (br s, 4H), HPLC: 8.37 min (100%), ESI-MS: m/z 411.1 [M+H]⁺

### Compound 6: [(1R,3S)-3-(3,4,5-trihydroxybenzoyl)oxycyclohexyl]3,4,5-trihydroxybenzoate

The compound was synthesized from cis-1,3-cyclohexanediol (2 mmol), 3,4,5-tris(benzyloxy)benzoic acid (4.2 mmol), DCC (4.6 mmol) and DMAP (4.4 mmol) as described above. Yield: 15% (120 mg, 0.29 mmol), ¹H-NMR, DMSO, 400 MHz: δ 1.34-1.47 (m, 3H), 1.49-1.58 (m, 1H), 1.82-1.86 (m, 1H), 1.98-2.01 (m, 2H), 2.37-2.40 (m, 1H), 4.85-4.91 (m, 2H), 6.95 (s, 4H), 8.93 (br s, 2H), 9.25 (br s, 4H), HPLC: 11.04 min (100%), ESI-MS: m/z 419.1 [M−H]⁻

### Compound 7: [2-hydroxy-3-(3,4,5-trihydroxybenzoyl)oxy-propyl]3,4,5-trihydroxybenzoate

The compound was synthesized from 2-benzyloxy-1,3-propanediol (2 mmol), 3,4,5-tris(benzyloxy)benzoic acid (4.2 mmol), DCC (4.6 mmol) and DMAP (4.4 mmol) as described above. Yield: 9% (70 mg, 0.18 mmol), ¹H-NMR, DMSO, 400 MHz: δ 4.06-4.11 (m, 1H), 4.19-4.27 (m, 4H), 6.99 (s, 4H), 8.96 (br s, 2H), 9.27 (br s, 4H), HPLC: 7.44 min (100%), ESI-MS: m/z 395.0 [M−H]⁻

### Compound 8: [2-amino-3-(3,4,5-trihydroxybenzoyl)oxy-propyl]3,4,5-trihydroxybenzoate

The compound was synthesized from 2-benzyloxy-1,3-propanediol (2 mmol), 3,4,5-tris(benzyloxy)benzoic acid (4.2 mmol), DCC (4.6 mmol) and DMAP (4.4 mmol) as described above. Yield: 5% (36 mg, 0.09 mmol), ¹H-NMR, DMSO, 400 MHz: δ 3.88-3.93 (m, 1H), 4.40-4.49 (m, 4H), 7.08 (s, 4H), 8.50 (br s, 3H), 9.28 (br s, 4H), HPLC: 6.03 min (97%), ESI-MS: m/z 396.2 [M+H]⁺

### Compound 9: 2,3-bis[(3,4,5-trihydroxybenzoyl)oxy]propyl 3,4,5-trihydroxybenzoate

The compound was synthesized from glycerol (1 mmol), 3,4,5-tris(benzyloxy)benzoic acid (3.1 mmol), DCC (3.3 mmol) and DMAP (3.2 mmol) as described above. Yield: 6% (33 mg, 0.06 mmol), ¹H-NMR, DMSO, 400 MHz: δ 4.45-4.58 (m, 4H), 5.53-5.58 (m, 1H), 6.95 (s, 6H), 8.60-9.60 (m, 9H), HPLC: 9.07 min (100%), ESI-MS: m/z 547.2 [M−H]⁻

### Compound 10: [3-(3,4,5-trihydroxybenzoyl)oxy-2-[(3,4,5-trihydroxybenzoyl)oxymethyl]propyl]3,4,5-trihydroxybenzoate

The compound was synthesized from 2-Hydroxymethyl-1,3-propanediol (2 mmol), 3,4,5-tris(benzyloxy)benzoic acid (6.4 mmol), DCC (6.8 mmol) and DMAP (6.6 mmol) as described above. Yield: 10% (105 mg, 0.19 mmol), ¹H-NMR, DMSO, 400 MHz: δ 2.67-2.73 (m, 1H), 4.37 (d, 6H, ³J=5.87 Hz), 6.99 (s, 6H), 8.99 (br s, 3H), 9.30 (br s, 6H), HPLC: 9.36 min (100%), ESI-MS: m/z 561.2 [M−H]⁻

### Compound 11: [2-methyl-3-(3,4,5-trihydroxybenzoyl)oxy-2-[(3,4,5-trihydroxybenzoyl)oxymethyl]-propyl]3,4,5-trihydroxybenzoate

The compound was synthesized from 1,1,1-Tris(hydroxymethyl)ethane (2 mmol), 3,4,5-tris(benzyl-oxy)benzoic acid (6.4 mmol), DCC (6.8 mmol) and DMAP (6.6 mmol) as described above. Yield: 1% (12 mg, 0.02 mmol), ¹H-NMR, DMSO, 400 MHz: δ 1.20 (s, 3H), 4.26 (s, 6H), 6.99 (s, 6H), 8.99 (br s, 3H), 9.30 (br s, 6H), HPLC: 9.95 min (100%), ESI-MS: m/z 575.2 [M−H]⁻

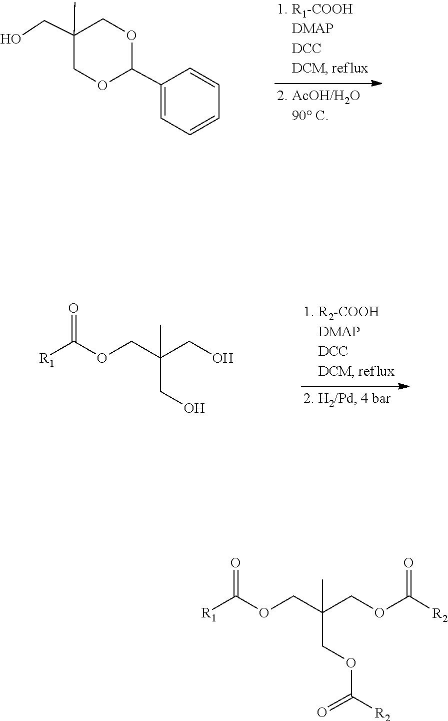

### Step 1

A solution of (5-methyl-2-phenyl-1,3-dioxan-5-yl)methanol (1 eq) in DCM (0.1 M) was treated with the respective benzoic acid derivative (1.1 eq), DCC (1.1 eq) and DMAP (1.1 eq) und heated to reflux overnight. The formed precipitate was removed by filtration and the filtrate was evaporated. The crude was purified by flash chromatography (silica, CHCl₃).

The residue was dissolved in AcOH/H2O (3:1, v/v; 0.1 M) and heated to 90° C. for 4 hours. The volatiles were evaporated and the crude was purified by flash chromatography (silica, heptane/ethylacetate gradient).

### Step 2

The respective monoester was dissolved in DCM (0.1 M), treated with benzoic acid derivative (2.2 eq), DCC (2.2 eq) and DMAP (2.2 eq) and heated to reflux overnight. The formed precipitate was removed by filtration and the filtrate was evaporated. The crude was purified by flash chromatography (silica, CHCl₃).

Deprotection of the phenol moieties was carried out by hydrogenation at 4 bar using Pd/C in CHCl₃/MeOH (1:1, v/v). After completion, the solution was filtered through celite and evaporated. The crude was purified by semi-preparative HPLC.

### Compound 12: [2-(benzoyloxymethyl)-2-methyl-3-(3,4,5-trihydroxybenzoyl)oxy-propyl]3,4,5-trihydroxybenzoate

The compound was synthesized from (5-methyl-2-phenyl-1,3-dioxan-5-yl)methanol (6 mmol), benzoic acid (6.6 mmol), DCC (6.6 mmol) and DMAP (6.6 mmol) for step 1 and 3,4,5-Tris(benzyloxy)benzoic Acid (3.6 mmol), DCC (3.6 mmol) and DMAP (3.6 mmol) for step 2 as described above. Yield: 7% (230 mg, 0.44 mmol), ¹H-NMR, DMSO, 400 MHz: δ 1.22 (s, 3H), 4.32-4.36 (m, 6H), 6.99 (s, 4H), 7.49-7.53 (m, 2H), 7.65-7.68 (m, 1H), 7.98-8.00 (m, 2H), 8.84-9.46 (m, 6H), HPLC: 13.28 min (100%)

### Compound 13: [3-benzoyloxy-2-(benzoyloxymethyl)-2-methyl-propyl]3,4,5-trihydroxybenzoate

The compound was synthesized from (5-methyl-2-phenyl-1,3-dioxan-5-yl)methanol (6 mmol), 3,4,5-Tris(benzyloxy)benzoic Acid (6.6 mmol), DCC (6.6 mmol) and DMAP (6.6 mmol) for step 1 and benzoic Acid (5.2 mmol), DCC (5.2 mmol) and DMAP (5.2 mmol) for step 2 as described above. Yield: 16% (445 mg, 0.93 mmol), ¹H-NMR, DMSO, 400 MHz: δ 1.24 (s, 3H), 4.37-4.41 (m, 6H), 6.99 (s, 2H), 7.48-7.52 (m, 4H), 7.64-7.68 (m, 2H), 7.97-7.99 (m, 4H), 8.88-9.42 (m, 3H), HPLC: 17.68 min (100%)

### Synthesis of Amide Derivatives

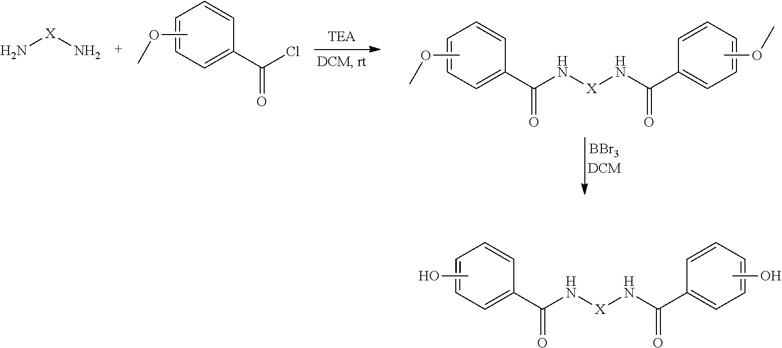

### Step 1

The respective amine (1 eq) was dissolved in DCM and cooled to 0° C. The triethylamine (2.1 eq) and 3,4,5-trimethoxybenzoyl chloride (2.1 equiv) were added portionwise. The mixture was stirred at 0° C. for one hour, then allowed to warm up to room temperature and stirring was continued overnight. The reaction was quenched by addition of water. The organic layer was separated and the aqueous layer was extracted twice with DCM. The combined organic layers were dried over Na₂SO₄ and evaporated. The crude was purified by flash chromatography (silica, CHCl₃/MeOH gradient).

### Step 2

The compound was dissolved in DCM and cooled to 0° C. BBr₃ (9 eq, 1 M solution in DCM) was added dropwise. After stirring at room temperature overnight, the reaction was quenched by addition of water and extracted with EtOAc. The combined organic layers were dried over Na₂SO₄ and evaporated. The crude was purified by semi-preparative HPLC.

For the synthesis of the triamides, 3.3 eq of the respective benzoylchloride and 3.3 eq of TEA were used in the first step.

### Compound 14: 3,4,5-trihydroxy-N-[3-[(3,4,5-trihydroxybenzoyl)amino]propyl]benzamide

The compound was synthesized from 1,3-diaminopropan (1.5 mmol) and 3,4,5-trimethoxybenzoyl chloride (3.2 mmol) as described above. Yield: 3% (12 mg, 0.03 mmol), ¹H-NMR, DMSO, 400 MHz: δ 1.66-1.70 (quin, 2H, ³J=6.8 Hz), 3.20-3.24 (m, 4H), 6.83 (s, 4H), 8.07-8.10 (m, 2H), 8.50-9.60 (m, 6H), HPLC: 5.76 min (100%), ESI-MS: m/z 377.2.1 [M−H]⁻

### Compound 15: 3,4,5-trihydroxy-N-[5-[(3,4,5-trihydroxybenzoyl)amino]pentyl]benzamide

The compound was synthesized from cadaverin (1.5 mmol) and 3,4,5-trimethoxybenzoyl chloride (3.2 mmol) as described above. Yield: 3% (17 mg, 0.04 mmol), ¹H-NMR, DMSO, 400 MHz: δ 1.28-1.33 (m, 2H), 1.46-1.54 (m, 4H), 3.14-3.19 (m, 4H), 6.81 (s, 4H), 8.02-8.05 (m, 2H), 8.59 (s, 2H), 8.98 (s, 4H), HPLC: 6.69 min (100%), ESI-MS: m/z 407.1 [M+H]⁺

### Compound 16: 3,4,5-trihydroxy-N-[10-[(3,4,5-trihydroxybenzoyl)amino]decyl]benzamide

The compound was synthesized from 1,10-diaminodecane (1.5 mmol) and 3,4,5-trimethoxybenzoyl chloride (3.2 mmol) as described above. Yield: 13% (95 mg, 0.2 mmol), ¹H-NMR, DMSO, 400 MHz: δ 1.22-1.33 (m, 12H), 1.45-1.48 (m, 4H), 3.13-3.18 (m, 4H), 6.81 (s, 4H), 8.00 (m, 2H), 8.61 (br s, 2H), 8.98 (br s, 4H), HPLC: 11.14 min (100%), ESI-MS: m/z 475.1 [M−H]⁻

### Compound 17: 3,4,5-trihydroxy-N-[2-[(3,4,5-trihydroxybenzoyl)-[2-[(3,4,5-trihydroxybenzoyl)-amino]-ethyl]amino]ethyl]benzamide

The compound was synthesized from diethylenetriamine (1.5 mmol) and 3,4,5-trimethoxybenzoyl chloride (5 mmol) as described above. Yield: 5% (39 mg, 0.07 mmol), ¹H-NMR, DMSO, 400 MHz: δ 4.28-4.61 (m, 8H), 7.51-7.78 (m, 2H), 8.32 (s, 2H), 8.71 (s, 4H), 9.48-9.78 (m, 3H), 9.78-10.18 (m, 6H), HPLC: 5.55 min (100%), ESI-MS: m/z 558.0 [M−H]⁻

### Compound 18: 3,4,5-trihydroxy-N-[2-methyl-3-[(3,4,5-trihydroxybenzoyl)amino]-2-[[(3,4,5-trihydroxy-benzoyl)amino]methyl]propyl]benzamide

The compound was synthesized from 2-(aminomethyl)-2-methyl-propane-1,3-diamine (1 mmol) and 3,4,5-trimethoxybenzoyl chloride (3.3 mmol) as described above. Yield: 18% (104 mg, 0.18 mmol), ¹H-NMR, DMSO, 400 MHz: δ 0.78 (s, 3H), 3.07-3.09 (m, 6H), 6.91 (s, 6H), 8.32-8.36 (m, 3H), 8.72 (br s, 1H), 9.13 (br s, 4H), HPLC: 7.68 min (>99%)

### Compound 19: 4-hydroxy-N-[3-[(4-hydroxybenzoyl)amino]-2-[[(4-hydroxybenzoyl)amino]methyl]-2-methyl-propyl]benzamide

The compound was synthesized from 2-(aminomethyl)-2-methyl-propane-1,3-diamine (1 mmol) and 4-methoxybenzoyl chloride (3.3 mmol) as described above. Yield: 16% (75 mg, 0.16 mmol), ¹H-NMR, DMSO, 400 MHz: δ 0.84 (s, 3H), 3.16 (d, 6H, ³J=6.4 Hz), 6.86 (d, 6H, ³J=8.7 Hz), 7.79 (d, 6H, ³J=8.7 Hz), 8.49 (t, 3H, ³J=6.4 Hz), 10.05 (br s, 3H), HPLC: 11.41 min (>99%); ESI-MS: m/z 478.5 [M+H]⁺

### Compound 20: 3-hydroxy-N-[3-[(3-hydroxybenzoyl)amino]-2-[[(3-hydroxybenzoyl)amino]methyl]-2-methyl-propyl]benzamide

The compound was synthesized from 2-(aminomethyl)-2-methyl-propane-1,3-diamine (1 mmol) and 3-methoxybenzoyl chloride (3.3 mmol) as described above. Yield: 13% (61 mg, 0.13 mmol), ¹H-NMR, DMSO, 400 MHz: δ 0.85 (s, 3H), 3.20 (d, 6H, ³J=6.6 Hz), 6.94-6.98 (m, 3H), 7.29-7.34 (m, 9H), 8.58 (t, 3H, ³J=6.6 Hz), 9.73 (br s, 3H), HPLC: 12.91 min (>99%); ESI-MS: m/z 478.2 [M+H]⁺

### Compound 21: N-[3-(1,3-benzodioxole-5-carbonylamino)-2-[(1,3-benzodioxole-5-carbonylamino)-methyl]-2-methyl-propyl]-1,3-benzodioxole-5-carboxamide

The compound was synthesized from 2-(aminomethyl)-2-methyl-propane-1,3-diamine (1 mmol) and Piperonyloyl chloride (3.3 mmol) as described above. Yield: 3% (17 mg, 0.03 mmol), ¹H-NMR, DMSO, 400 MHz: δ 0.85 (s, 3H), 3.19 (d, 6H, ³J=6.5 Hz), 6.13 (s, 6H), 7.05 (d, 3H, ³J=8.2 Hz), 7.42 (d, 3H, ⁴J=1.7 Hz), 7.50 (dd, 3H, ³J=8.2 Hz, ⁴J=1.7 Hz), 8.51 (t, 3H, ³J=6.5 Hz), HPLC: 17.89 min (>99%); ESI-MS: m/z 562.4 [M+H]⁺

### Compound 22: N-[3-[(2,4-dihydroxybenzoyl)amino]-2-[[(2,4-dihydroxybenzoyl)amino]methyl]-2-methyl-propyl]-2,4-dihydroxy-benzamide

The compound was synthesized from 2-(aminomethyl)-2-methyl-propane-1,3-diamine (1 mmol) and 2,4-dimethoxybenzoic acid (3 mmol). Amide coupling was performed using TBTU (3.3 mmol) and DIPEA (4.5 mmol) in DMF (5 ml) at room temperature overnight. The reaction was quenched with water and diluted HCl and extracted with EtOAc. The combined organic layers were dried over Na₂SO₄ and evaporated. The crude was purified by flash chromatography (silica, CHCl₃/MeOH gradient). Ether deprotection was carried out as described above. Yield: 6% (31 mg, 0.06 mmol), ¹H-NMR, DMSO, 400 MHz: δ 0.85 (s, 3H), 3.21 (d, 6H, ³J=6.5 Hz), 6.30 (d, 3H, ⁴J=2.4 Hz), 6.36 (dd, 3H, ³J=8.7 Hz, ⁴J=2.4 Hz), 7.73 (d, 3H, ³J=8.7 Hz), 8.73 (t, 3H, ³J=6.5 Hz), 10.09 (br s, 3H), 12.37 (br s, 3H), HPLC: 14.29 min (>99%); ESI-MS: m/z 526.4 [M+H]⁺

### Compound 23: N-[3-[(3,4-dihydroxybenzoyl)amino]-2-[[(3,4-dihydroxybenzoyl)amino]methyl]-2-methyl-propyl]-3,4-dihydroxy-benzamide

The compound was synthesized from 2-(aminomethyl)-2-methyl-propane-1,3-diamine (1 mmol) and 3,4-dimethoxybenzoic acid (3 mmol). Amide coupling was performed using TBTU (3.3 mmol) and DIPEA (4.5 mmol) in DMF (5 ml) at room temperature overnight. The reaction was quenched with water and diluted HCl and extracted with EtOAc. The combined organic layers were dried over Na₂SO₄ and evaporated. The crude was purified by flash chromatography (silica, CHCl₃/MeOH gradient). Ether deprotection was carried out as described above. Yield: 16% (86 mg, 0.16 mmol), ¹H-NMR, DMSO, 400 MHz: δ 0.80 (s, 3H), 3.11 (d, 6H, ³J=6.5 Hz), 6.82 (d, 3H, ³J=8.2 Hz), 7.26 (dd, 3H, ³J=8.2 Hz, ⁴J=2.1 Hz), 7.36 (d, 3H, ⁴J=2.1 Hz), 8.42 (t, 3H, ³J=6.5 Hz), 9.21 (br s, 3H), 9.52 (br s, 3H), HPLC: 9.52 min (96.9%); ESI-MS: m/z 526.5 [M+H]⁺

### Compound 24: N-[3-[(2,3-dihydroxybenzoyl)amino]-2-[[(2,3-dihydroxybenzoyl)amino]methyl]-2-methyl-propyl]-2,3-dihydroxy-benzamide

The compound was synthesized from 2-(aminomethyl)-2-methyl-propane-1,3-diamine (1 mmol) and 2,3-dimethoxybenzoic acid (3 mmol). Amide coupling was performed using TBTU (3.3 mmol) and DIPEA (4.5 mmol) in DMF (5 ml) at room temperature overnight. The reaction was quenched with water and diluted HCl and extracted with EtOAc. The combined organic layers were dried over Na₂SO₄ and evaporated. The crude was purified by flash chromatography (silica, CHCl₃/MeOH gradient). Ether deprotection was carried out as described above. Yield: 23% (120 mg, 0.23 mmol), ¹H-NMR, DMSO, 400 MHz: δ 0.90 (s, 3H), 3.29 (d, 6H, ³J=6.4 Hz), 6.67 (t, 3H, ³J=8.0 Hz), 6.96 (dd, 3H, ³J=8.0 Hz, ⁴J=1.4 Hz), 7.31 (dd, 3H, ³J=8.0 Hz, ⁴J=1.4 Hz), 8.87 (t, 3H, ³J=6.4 Hz), 9.30 (br s, 3H), 12.05 (br s, 3H), HPLC: 14.93 min (>99%); ESI-MS: m/z 526.5 [M+H]⁺

### Example 2: In Vitro Screening of Small-Molecule Compounds Against RSV and SARS-CoV-2

Compounds listed in Table 2 were synthesized as described above, screened for antiviral activity against RSV using an established in vitro RSV inhibitory assay, and displayed concentration-dependent inhibition of virus infection in the nanomolar concentration range. Compounds were also tested against SARS-CoV-2 in viral inhibition assay. Treatment of cells with compounds at nanomolar concentrations led to concentration-dependent inhibition of SARS-CoV-2 infection.

Dose-response curves for identified inhibitors against RSV and SARS-CoV-2, and for in vitro cytotoxicity analysis are exemplified in FIGS. 1 to 6 and 9, where the numbers of the respective tested compound are given. The results are summarized in Table 2.

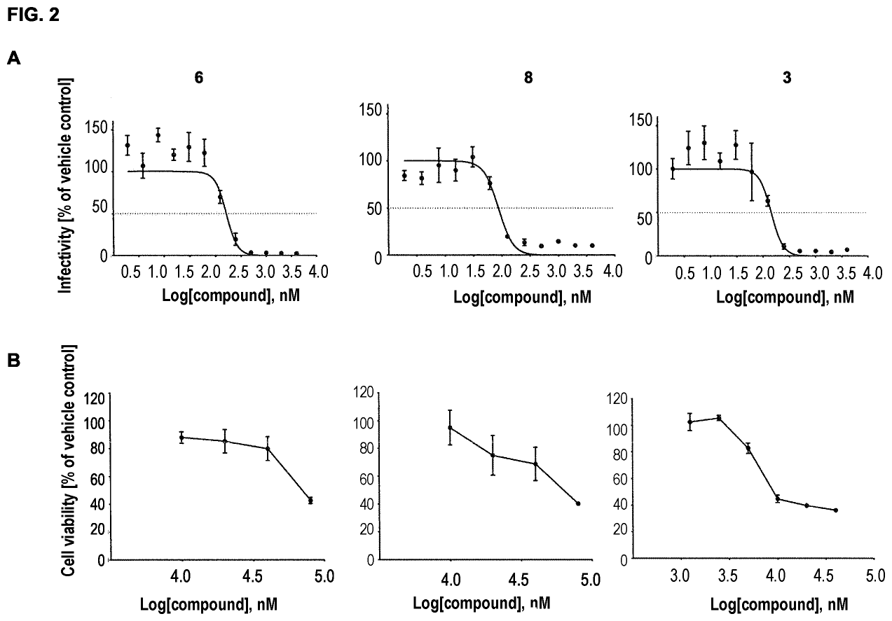

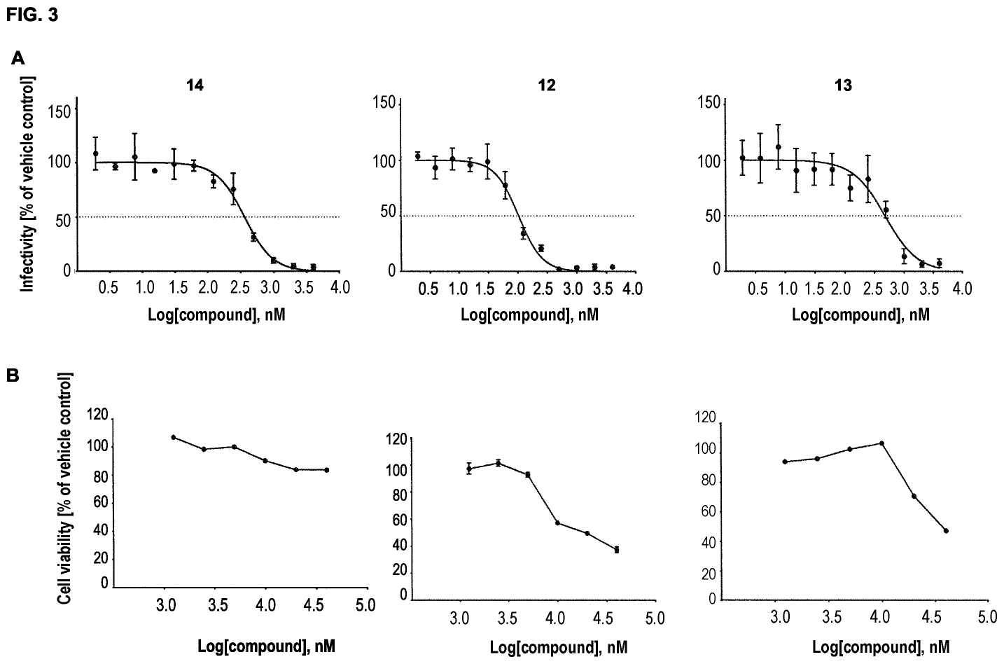

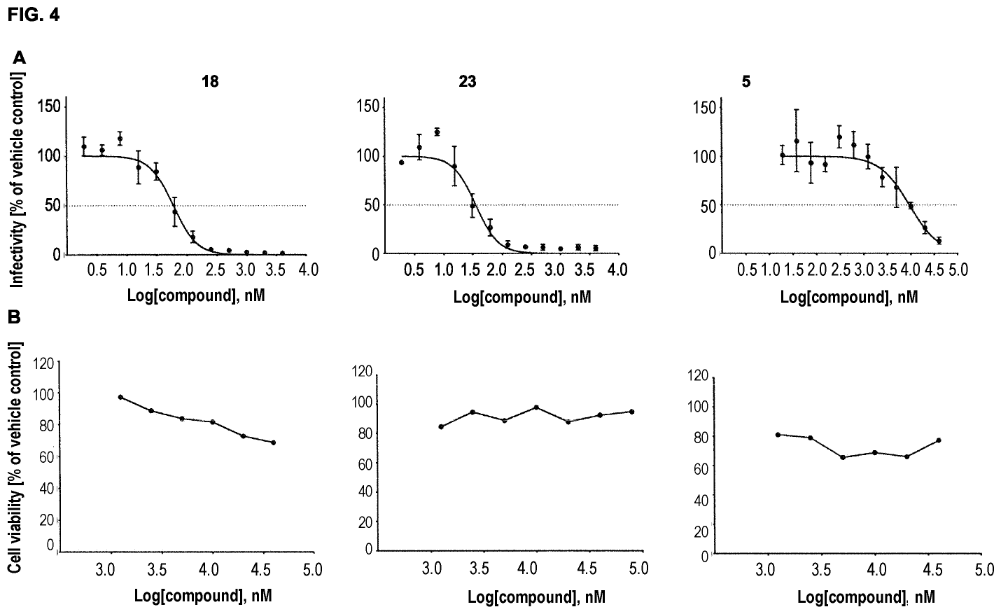

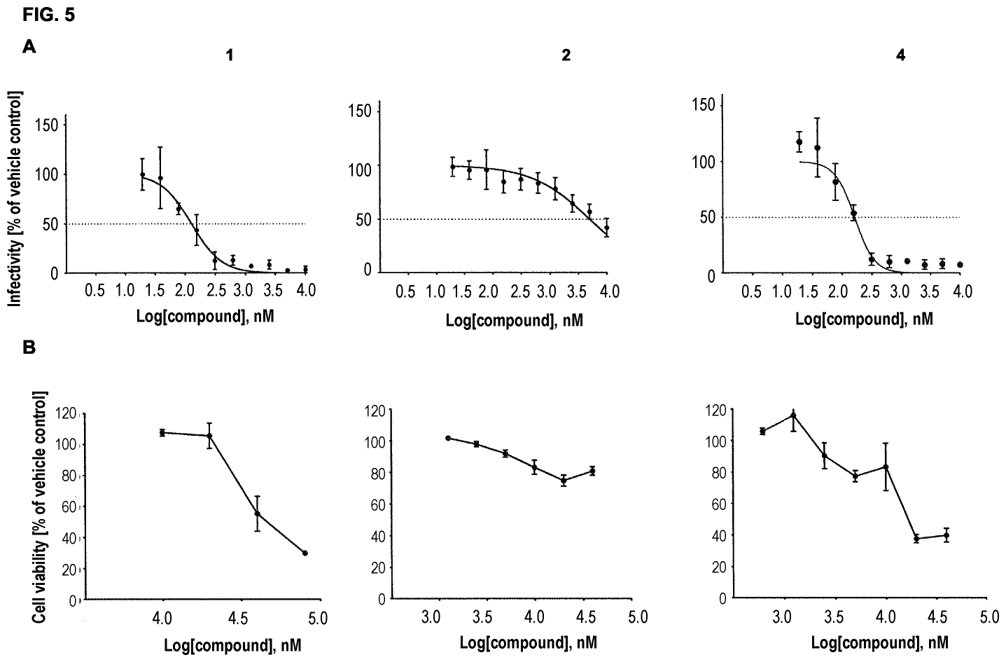

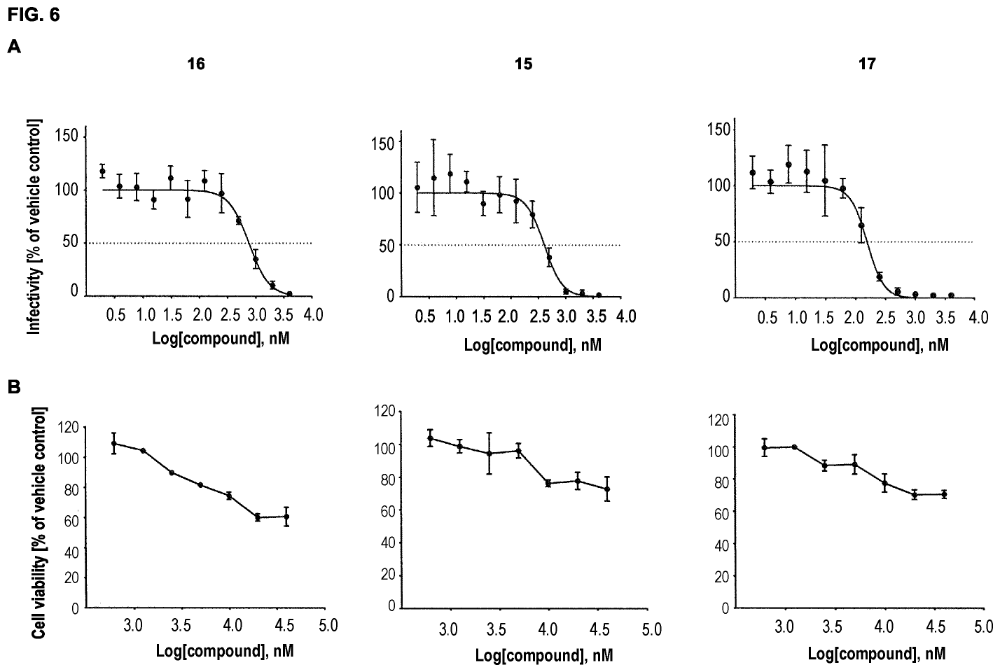

FIG. 1A, FIG. 2A, FIG. 3A, FIG. 4A, FIG. 5A, and FIG. 6A show dose-response curves for identified inhibitors against RSV. RSV was incubated with increasing concentrations of compounds for 10 min at 37° C. before the compound-virus mixtures were added to human epithelial cells (HEp-2). 2 dpi, infection rates were measured by counting infected cells. Vehicle-treated cells were set as the reference at 100% and used to normalize the data. Shown values represent mean±SD (n=3). The IC50 was determined by fitting the data to a dose-response curve using nonlinear regression analysis (GraphPad Prism, version 6.02). FIG. 1B, FIG. 2B, FIG. 3B, FIG. 4B, FIG. 5B, and FIG. 6B show dose-response curves for identified inhibitors (the same as identified at the top of the respective figures) in in vitro cytotoxicity analysis. HEp-2 cells were treated with increasing concentration of compounds without RSV-infection to assess potential cytotoxicity of the compounds. 48 h post-treatment cells were analyzed using CellTiter-Glo® Luminescent Cell Viability Assay (Promega) to determine cell viability rates. Vehicle-treated cells were set as the reference at 100% of viability and used to normalize the data. Shown values represent mean+SD (n=3).

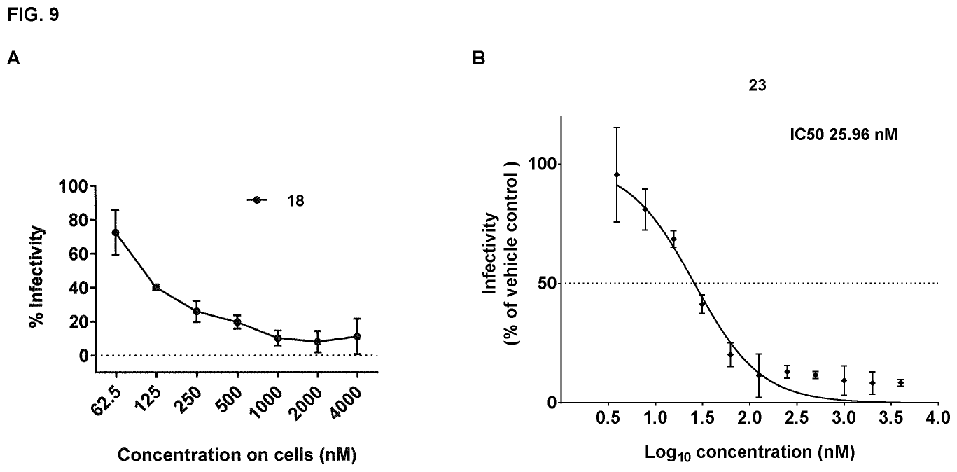

FIG. 9 shows antiviral activity of exemplary small molecule compounds against SARS-CoV-2 using cell culture-based infection model. (A): SARS-CoV-2 (German isolate) was incubated with increasing concentrations of either compound 18 or corresponding concentration of DMSO-vehicle control for 10 min at 37° C. before the compound-virus mixtures were added to Vero E6 cells. The cells were fixed and stained using anti-SARS-CoV-2-S antibody and a secondary peroxidase-labeled goat anti-IgG. Cell foci were visualized using TrueBlue peroxidase substrate. Shown values represent mean±SD (n=3). (B): One day before infection, 20000 Vero E6 cells/well were seeded in 96-well plates. The next day, compounds were mixed with infectious SARS-CoV-2 and incubated for 10 min at 37° C. After incubation, compound-virus mixtures were applied to cells in triplicates at indicated concentrations. Infection rates were analyzed 27 hours post-infection by immunocytochemical staining using mAb against SARS-CoV2-S protein. Infectivity is shown as mean values of counted focus-forming units of triplicate wells normalized to mock-treated controls ±SD. Results for compound 23 are shown in this figure; results for further compounds are shown in Table 2.

TABLE 2
Compound numbers, structures, and test results

| | | | | Cell |
|---|---|---|---|---|
| | | | | viability |
| | | IC₅₀ [nM] | | on |
| | | | SARS- | HEp-2 |
| Compound | Structure | RSV | CoV-2 | cells |
| 1 | 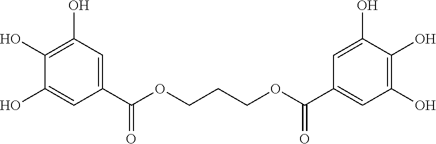 [SMILES: O=C(OCCCOC(=O)c1cc(O)c(O)c(O)c1)c1cc(O)c(O)c(O)c1] | 128.9 | | 72737 |
| 2 | 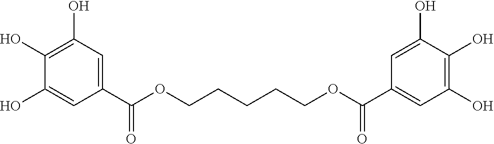 [SMILES: O=C(OCCCCCOC(=O)c1cc(O)c(O)c(O)c1)c1cc(O)c(O)c(O)c1] | 4959 | | >80000 |
| 3 | 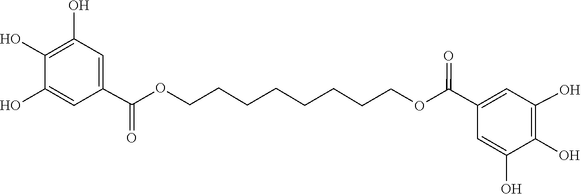 [SMILES: O=C(OCCCCCCCCOC(=O)c1cc(O)c(O)c(O)c1)c1cc(O)c(O)c(O)c1] | 145 | | 9197 |
| 4 | 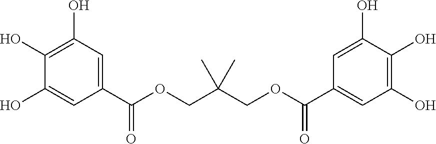 [SMILES: CC(C)(COC(=O)c1cc(O)c(O)c(O)c1)COC(=O)c1cc(O)c(O)c(O)c1] | 162.2 | | 63163 |
| 5 | 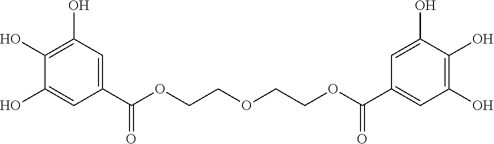 [SMILES: O=C(OCCOCCOC(=O)c1cc(O)c(O)c(O)c1)c1cc(O)c(O)c(O)c1] | 9334 | | 638066 |
| 6 | 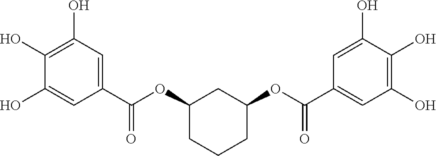 [SMILES: O=C(O[C@@H]1CCC[C@H](OC(=O)c2cc(O)c(O)c(O)c2)C1)c1cc(O)c(O)c(O)c1] | 165.1 | | 61709 |
| 7 | 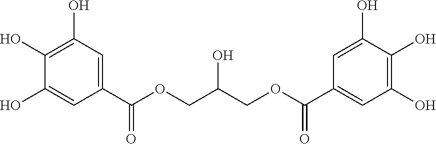 [SMILES: O=C(OCC(O)COC(=O)c1cc(O)c(O)c(O)c1)c1cc(O)c(O)c(O)c1] | 339.2 | | >80000 |
| 8 | 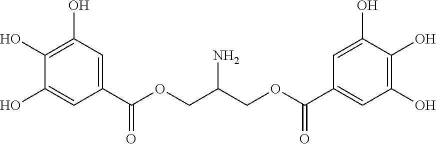 [SMILES: NC(COC(=O)c1cc(O)c(O)c(O)c1)COC(=O)c1cc(O)c(O)c(O)c1] | 87.7 | | 66234 |
| 9 | 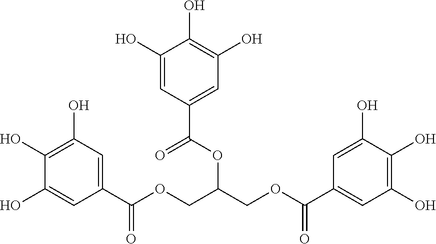 [SMILES: O=C(OCC(COC(=O)c1cc(O)c(O)c(O)c1)OC(=O)c1cc(O)c(O)c(O)c1)c1cc(O)c(O)c(O)c1] | 55.49 | | 49099.5 |
| 10 | 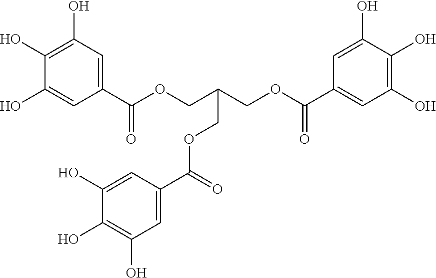 [SMILES: O=C(OCC(COC(=O)c1cc(O)c(O)c(O)c1)COC(=O)c1cc(O)c(O)c(O)c1)c1cc(O)c(O)c(O)c1] | 47.21 | | 75145 |
| 11 | 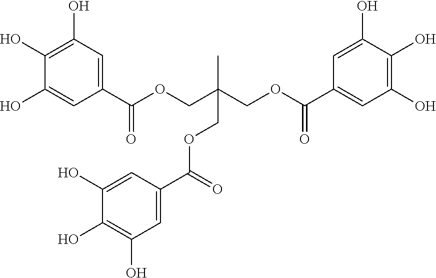 [SMILES: CC(COC(=O)c1cc(O)c(O)c(O)c1)(COC(=O)c1cc(O)c(O)c(O)c1)COC(=O)c1cc(O)c(O)c(O)c1] | 36.73 | | 47303 |
| 12 | 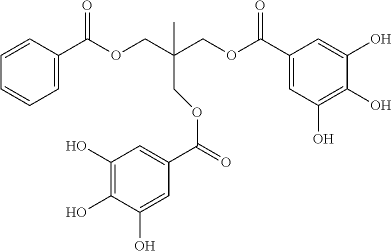 [SMILES: CC(COC(=O)c1ccccc1)(COC(=O)c1cc(O)c(O)c(O)c1)COC(=O)c1cc(O)c(O)c(O)c1] | 104.7 | | 34098.5 |
| 13 | 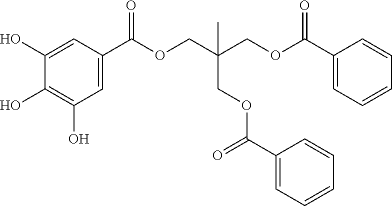 [SMILES: CC(COC(=O)c1ccccc1)(COC(=O)c1ccccc1)COC(=O)c1cc(O)c(O)c(O)c1] | 480.5 | | 13042 |
| 14 | 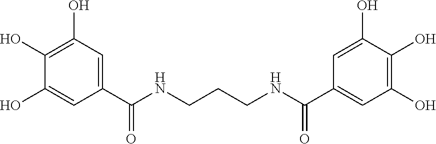 [SMILES: O=C(NCCCNC(=O)c1cc(O)c(O)c(O)c1)c1cc(O)c(O)c(O)c1] | 367.3 | | 15677 |
| 15 | 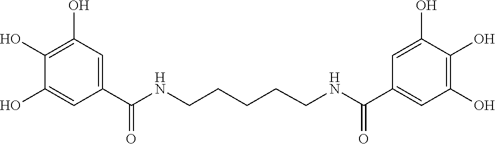 [SMILES: O=C(NCCCCCNC(=O)c1cc(O)c(O)c(O)c1)c1cc(O)c(O)c(O)c1] | 408.2 | | 45401 |
| 16 | 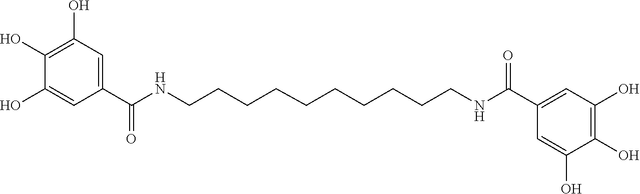 [SMILES: O=C(NCCCCCCCCCCNC(=O)c1cc(O)c(O)c(O)c1)c1cc(O)c(O)c(O)c1] | 767 | | >80000 |
| 17 | 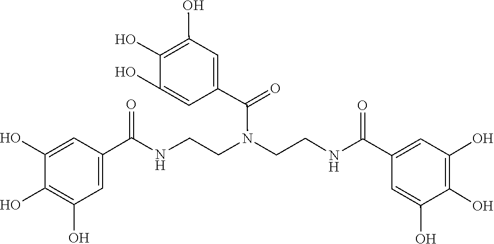 [SMILES: O=C(NCCN(CCNC(=O)c1cc(O)c(O)c(O)c1)C(=O)c1cc(O)c(O)c(O)c1)c1cc(O)c(O)c(O)c1] | 155.4 | | 36216 |
| 18 | 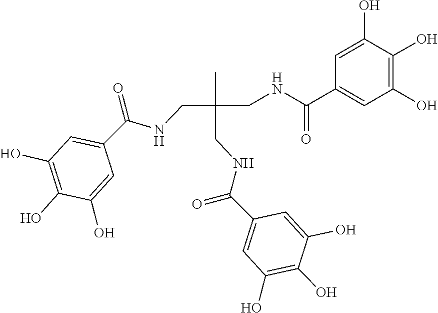 [SMILES: CC(CNC(=O)c1cc(O)c(O)c(O)c1)(CNC(=O)c1cc(O)c(O)c(O)c1)CNC(=O)c1cc(O)c(O)c(O)c1] | 59.5 | | >80000 |
| 19 | 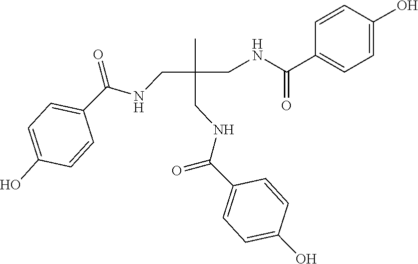 [SMILES: CC(CNC(=O)c1ccc(O)cc1)(CNC(=O)c1ccc(O)cc1)CNC(=O)c1ccc(O)cc1] | | | >80000 |
| 20 | 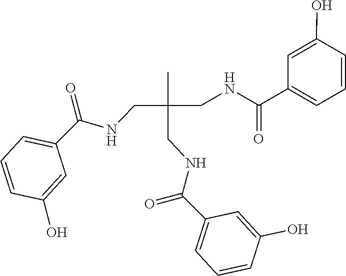 [SMILES: CC(CNC(=O)c1cccc(O)c1)(CNC(=O)c1cccc(O)c1)CNC(=O)c1cccc(O)c1] | | | 76260 |
| 21 | 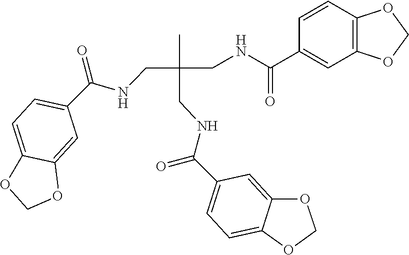 [SMILES: CC(CNC(=O)c1ccc2c(c1)OCO2)(CNC(=O)c1ccc2c(c1)OCO2)CNC(=O)c1ccc2c(c1)OCO2] | | | >80000 |
| 22 | 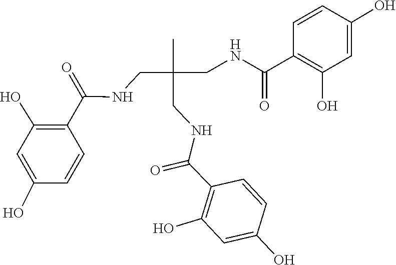 [SMILES: CC(CNC(=O)c1ccc(O)cc1O)(CNC(=O)c1ccc(O)cc1O)CNC(=O)c1ccc(O)cc1O] | | 75.13 | 17473 |
| 23 | 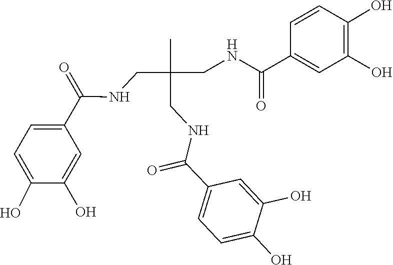 [SMILES: CC(CNC(=O)c1ccc(O)c(O)c1)(CNC(=O)c1ccc(O)c(O)c1)CNC(=O)c1ccc(O)c(O)c1] | 35.67 | 25.96 | >80000 |
| 24 | 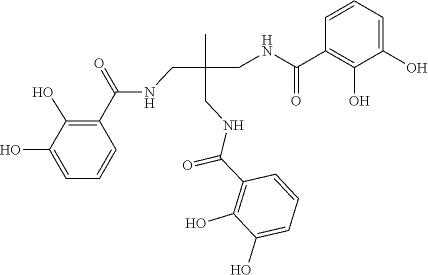 [SMILES: CC(CNC(=O)c1cccc(O)c1O)(CNC(=O)c1cccc(O)c1O)CNC(=O)c1cccc(O)c1O] | | | 5822.5 |
| 4,5-DCQA* | 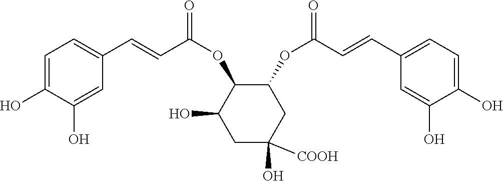 [SMILES: O=C(/C=C/c1ccc(O)c(O)c1)O[C@@H]1[C@H](O)C[C@@](O)(C(=O)O)C[C@H]1OC(=O)/C=C/c1ccc(O)c(O)c1] | 1093 | 217.6 | >80000 |

* reference compound

### Example 3: Targeting Viral Particles

To identify the site of action of designed inhibitors, compounds were either incubated with virus and then applied to cells or incubated on the cells and washed out prior to infection.

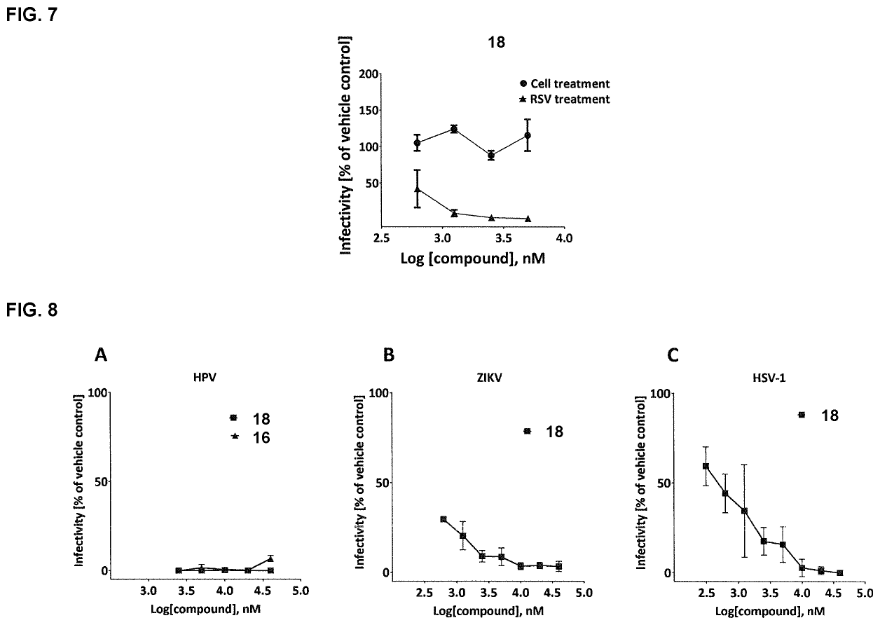

FIG. 7 shows the results of an experiment concerning the mechanism of inhibition of RSV infection. For virus treatment, RSV was incubated with increasing concentrations of the inhibitor for 10 min at 37° C. before the mixtures were added to HEp-2 cells. For cell treatment, the inhibitor was added directly to the cells; after 2 h, the medium was replaced, and the cells were infected with RSV. 48 h post-infection infection rates were measured. Vehicle-treated cells were set as the reference at 100% and used to normalize the data. Shown values represent mean±SD (n=3).

The results presented in FIG. 7 show that when the compounds are added to cells and then washed out before infection, no antiviral effect is observed. This suggests that the inhibitors act on RSV-virions rather than on the target cells.

### Example 4: Screening of Small-Molecule Compounds Against Other Viruses

The antiviral activity of the identified molecules was not limited to RSV or SARS-CoV-2. Other viruses including HSV-1, HPV and ZIKV can be inhibited by the compounds at similarly low concentrations, as shown in FIG. 8. Small molecule compounds were incubated at increasing concentrations for 10 min at 37° C. with either HPV (A), ZIKV (B) or HSV-1 (C). Compound-virus mixtures were added to HEK293T cells (A) or Vero E6 cells (B and C) and incubated for 48 h. Infection rate in vehicle-treated cells were set as the reference at 100% and used to normalize the data. Shown values represent mean±SD (n=3).

### Example 5: Stability in Solution

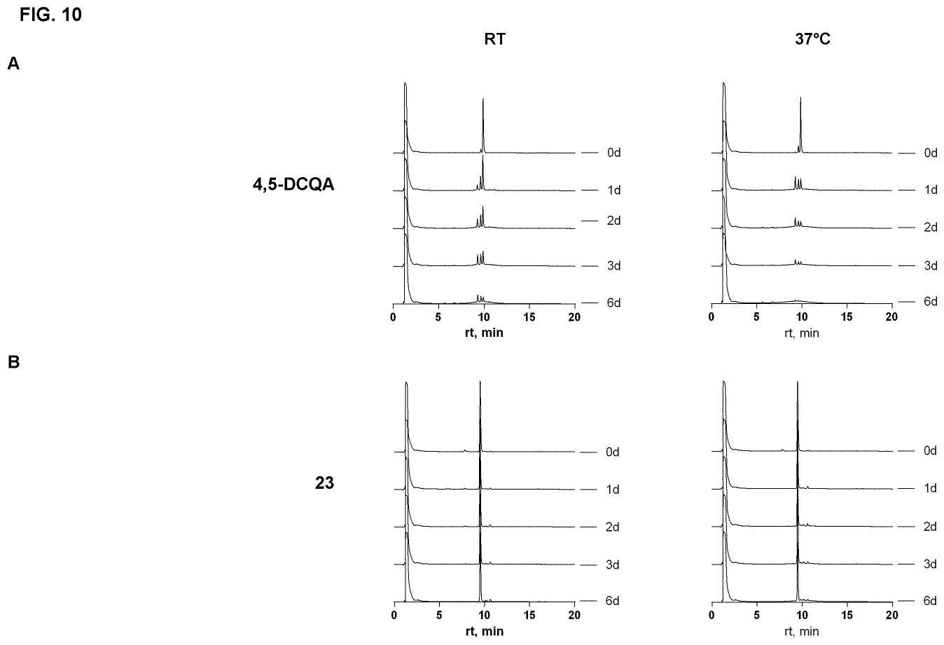

FIG. 10 shows improved stability of compound 23 in solution (500 μM in DMSO/PBS (5:95 v/v); incubation at room temperature (left) or 37° C. (right); (A): 4,5-DCQA (reference); (B): compound 23).

### Example 6: Combination of Peptide and Small Molecule Inhibitors

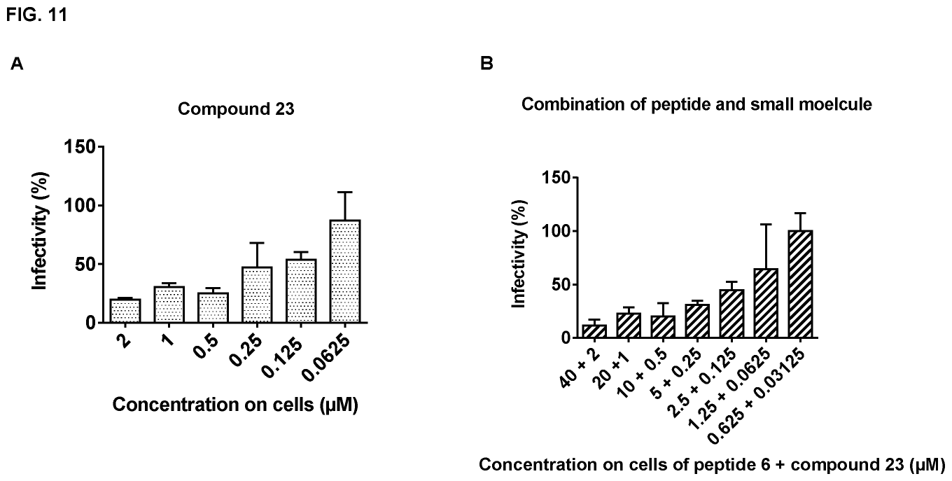

RSV was incubated with increasing concentrations either of small molecule compound 23 (FIG. 11A) or mixture of small molecule compound 23 and peptide-6 (Ac-[Chg][Cpa]VSTT[Tyr3.5-12]LPHYFDN-NH₂) (FIG. 11B) for 10 min at 37° C. before the compound-virus mixtures were added to human epithelial cells (HEp-2). 2 dpi, infection rates were measured by counting infected cells. Vehicle-treated cells were set as the reference at 100% and used to normalize the data. Shown values represent mean±SD (n=3). The combination shows excellent inhibitory effect.

The results reveal that the compounds, compositions and combinations have the potential to be effective and inhibit different viruses in vitro. The inhibitors can serve as a cost-effective therapeutic or prophylactic approach for population groups for which the benefits of vaccination are limited, such as immunocompromised patients and elderly.

1. A method for treating a viral infection caused by a virus belonging to a taxon selected from Alphaherpesvirinae; Monodnaviria; Negarnaviricota; Pisuviricota; and Flavivirus;
comprising administering a therapeutically effective amount of a compound, or a pharmaceutical composition comprising the compound, to a subject in need thereof, wherein the compound has a structure according to Formula (I), an enantiomer, a diastereoisomer, a hydrate, solvate, a crystal form, a tautomer, or a pharmaceutically acceptable salt thereof: 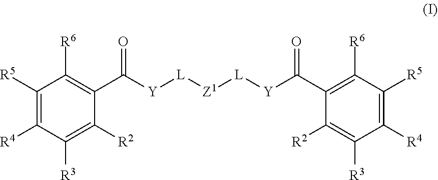
wherein:
L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
Y is selected, independently for each occurrence, from O and NR⁷;
Z¹ is selected from bond, O, NR, CH₂, CH(R), CH(R¹), and CR(R¹);
R is selected from OH, optionally substituted C₁₋₆ alkyl, NR⁷R⁸, and 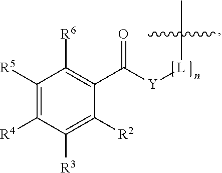
wherein n is 0 or 1;
R¹ is selected from OH, optionally substituted C₁₋₆ alkyl, and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, or R⁶ is OH, and/or wherein at least two occurrences of R², R³, R⁴, R⁵, or R⁶ form together part of an optionally substituted 4- to 8-membered heterocyclic ring containing 0; and
R⁷ and R⁸ are selected, independently for each occurrence, from H and optionally substituted C₁₋₆ alkyl.

2. The method according to claim 1, wherein the virus belongs to a taxon selected from:
(a) Alphaherpesvirinae; Shotokuvirae; Negarnaviricota; Pisuviricota; and Flavivirus;
(b) Alphaherpesvirinae; Cossaviricota; Haploviricotina; Pisuviricota; and Flavivirus;
(c) preferably: Alphaherpesvirinae; Papovaviricetes; Monjiviricetes; Pisoniviricetes; and Flavivirus;
(d) Alphaherpesvirinae; Zurhausenvirales; Mononegavirales; Nidovirales-preferably, Cornidovirineae; and Flavivirus;
(e) Alphaherpesvirinae; Papillomaviridae; Firstpapillomavirinae or Secondpapillomavirinae; Pneumoviridae; Coronaviridae; Orthocoronavirinae; and Flavivirus; or
(f) Simplexvirus; Alphapapillomavirus; Orthopneumovirus; Betacoronavirus; Sarbecovirus; and Flavivirus.

3. The method according to claim 1, wherein
L is, independently for each occurrence, C₁₋₆ alkylene, preferably linear or branched C₁, C₂, C₃, C₄, C₅ or C₅ alkylene, or methylene, each of which can be optionally substituted with one or more of halogen, OH, CN, NR⁷R⁸, C(O)NR⁷R⁸, C(O)OR⁷, and OR⁷,
wherein carbon atoms from two L groups can optionally form, together with Z¹, part of a 4- to 8-membered carbocyclic or heterocyclic ring optionally substituted with one or more of halogen, OH, CN, NR⁷R⁸, C(O)NR⁷R⁸, C(O)OR⁷, and OR⁷;
Y is selected, independently for each occurrence, from O and NR⁷;
Z¹ is selected from bond, O, NR, CH₂, CH(R), CH(R¹), and CR(R¹);
R is selected from:
OH,
linear or branched, acyclic or cyclic C₁₋₆ alkyl, C₁₋₃ alkyl, or methyl, each of which can be optionally substituted with one or more of halogen, OH, CN, NR⁷R⁸, C(O)NR⁷R⁸, C(O)OR⁷, and OR⁷,
NR⁷R⁸, and 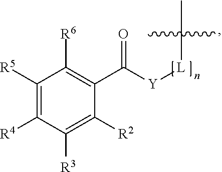
wherein n is 0 or 1;
R¹ is selected from:
OH,
linear or branched, acyclic or cyclic C₁₋₆ alkyl, C₁₋₃ alkyl or methyl, each of which can be optionally substituted with one or more of halogen, OH, CN, NR⁷R⁸, C(O)NR⁷R⁸, C(O)OR⁷, and OR⁷, and
NR⁷R⁸;
R², R³, R⁴, R⁵, and R⁶ are selected, independently for each occurrence, from:
H, halogen, OH, CN, NR⁷R⁸, C(O)NR⁷R⁸, and OR⁷,
wherein at least one occurrence of R², R³, R⁴, R⁵, or R⁶ is OH, and/or wherein at least two occurrences of R², R³, R⁴, R⁵, and R⁶ form together part of a 4- to 8-membered heterocyclic ring containing O which can be optionally substituted with one or more of halogen, OH, CN, NR⁷R⁸, OR⁷, C(O)NR⁷R⁸, C(O)OR⁷, and OR⁷; and
R⁷ and R⁸ are selected, independently for each occurrence, from H, and linear or branched, acyclic or cyclic C₁₋₆ alkyl, C₁₋₃ alkyl or methyl, each of which can be optionally substituted with one or more of halogen, OH, CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)OH, C(O)O(C₁₋₆ alkyl), and O(C₁₋₆ alkyl), wherein each C₁₋₆ alkyl is independently selected and can be optionally substituted with one or more of halogen, OH, CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)OH, C(O)O(C₁₋₆ alkyl), and O(C₁₋₆ alkyl).

4. The method according to claim 1, wherein Z is selected from O, CH(R), CH(R¹), and CR(R¹).

5. The method according to claim 1, wherein:
Z¹ is selected from CH(R¹) and CR(R¹); and
R¹ is selected from optionally substituted C₁₋₆ alkyl and NR⁷R⁸.

6. The method according to claim 1, wherein:
(a) at least one occurrence of Y is NR⁷; and Z¹ is selected from CH(R¹) and CR(R¹); or
(b) at least one occurrence of Y is O; and Z is selected from O, NR, CH₂, CH(R), and CH(R¹).

7. The method according to claim 1, wherein:
(a) on at least one of the phenyl rings in Formula (I), each of R³ and R⁴ is OH;
(b) on at least one of the phenyl rings in Formula (I), each of R³ and R⁴ is OH, and each of R², R⁵, and R⁶ is H or OH:
(c) on at least one of the phenyl rings in Formula (I), each of R³ and R⁵ is OH; or
(d) on at least one of the phenyl rings in Formula (I), each of R³ and R⁵ is OH, and each of R², R⁴, and R⁶ is H or OH.

8. The method according to claim 1, wherein on at least one, on at least two, or on three of the phenyl rings in Formula (I), three of R², R³, R⁴, R⁵, and R⁶ are OH.

9. A method for treating a viral infection, comprising administering a therapeutically effective amount of a compound, or a pharmaceutical composition comprising the compound, to a subject in need thereof,
wherein the compound has a structure according to Formula (I) as defined in claim 1, an enantiomer, a diastereoisomer, a hydrate, a solvate, a crystal form, a tautomer, or a pharmaceutically acceptable salt thereof; and
wherein in Formula (I):
L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
Y is selected, independently for each occurrence, from O and NR⁷;
Z¹ is selected from O, CH(R), CH(R¹), and CR(R¹);
R is selected from OH, optionally substituted C₁₋₆ alkyl, NR⁷R⁸, and 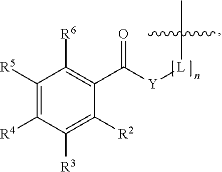
wherein n is 0 or 1;
R¹ is selected from OH, optionally substituted C₁₋₆ alkyl, and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, or R⁶ is OH, and/or wherein at least two occurrences of R², R³, R⁴, R⁵, or R⁶ form together part of an optionally substituted 4- to 8-membered heterocyclic ring containing O; and
R⁷ and R⁸ are selected, independently for each occurrence, from H and optionally substituted C₁₋₆ alkyl.

10. The method according to claim 9, wherein the viral infection is caused by a virus belonging to a taxon selected from:
(a) Duplodnaviria; Monodnaviria; and Riboviria;
(b) Heunggongvirae; Shotokuvirae; and Orthornavirae;
(c) Peploviricota; Cossaviricota; Negarnaviricota-Haploviricotina; Pisuviricota; and Kitrinoviricota;
(d) Herviviricetes; Papovaviricetes; Monjiviricetes; Pisoniviricetes; and Flasuviricetes;
(e) Herpesvirales; Zurhausenvirales; Mononegavirales; Nidovirales; Cornidovirineae; and Amarillovirales;
(f) Herpesviridae; Alphaherpesvirinae; Papillomaviridae; Firstpapillomavirinae or Secondpapillomavirinae; Pneumoviridae; Coronaviridae; Orthocoronavirinae; and Flaviviridae;
(g) Simplexvirus; Alphapapillomavirus; Orthopneumovirus; Betacoronavirus Sarbecovirus; and Flavivirus.

11. The method according to claim 1, wherein the virus is selected from Human alphaherpesvirus; Human papillomavirus; Respiratory syncytial virus; Severe acute respiratory syndrome-related coronavirus; and Zika virus.

12. The method according to claim 1, wherein the virus is selected from HSV-1; HPV-16; RSV; SARS-CoV-2; and Zika virus.

13. The method according to claim 1, wherein the virus is an enveloped virus.

14. A compound, or a pharmaceutical composition comprising the compound, wherein the compound has a structure according to Formula (I) as defined in claim 1, an enantiomer, a diastereoisomer, hydrate, a solvate, a crystal form, a tautomer, or a pharmaceutically acceptable salt thereof, and
wherein in Formula (I): 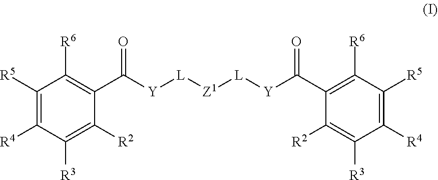
L is, independently for each occurrence, optionally substituted C₁₋₆ alkylene, wherein carbon atoms from two L groups can optionally form, together with Z¹, part of an optionally substituted 4- to 8-membered carbocyclic or heterocyclic ring;
Y is selected, independently for each occurrence, from O and NR⁷;
Z¹ is selected from CH(R), CH(R¹), and CR(R¹); wherein if all occurrences of Y are O, then Z¹ is selected from CH(R¹), and CR(R¹);
R is selected from OH, optionally substituted C₁₋₆ alkyl, NR⁷R⁸, and 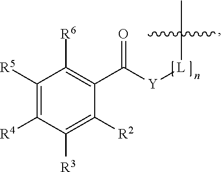
wherein n is 0 or 1;
R¹ is selected from optionally substituted C₁₋₆ alkyl and NR⁷R⁸;
R², R³, R⁴, R⁵, and R⁶ are selected, independently for each occurrence, from H, halogen, OH, CN, NR⁷R⁸, C(O)NR⁷R⁸, and OR⁷, wherein at least one occurrence of R², R³, R⁴, R⁵, and R⁶ is OH, and/or wherein at least two occurrences of R², R³, R⁴, R⁵, and R⁶ form together part of an optionally substituted 4- to 8-membered heterocyclic ring containing 0; and
R⁷ and R⁸ are selected, independently for each occurrence, from H and optionally substituted C₁₋₆ alkyl,
wherein the compound does not have the structure

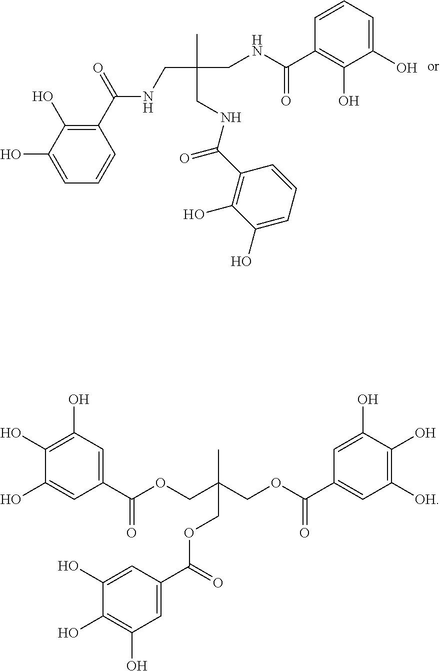 [SMILES: CC(CNC(=O)c1cccc(O)c1O)(CNC(=O)c1cccc(O)c1O)CNC(=O)c1cccc(O)c1O ; "or" ; CC(COC(=O)c1cc(O)c(O)c(O)c1)(COC(=O)c1cc(O)c(O)c(O)c1)COC(=O)c1cc(O)c(O)c(O)c1]

15. A pharmaceutical combination comprising: a compound having a structure according to Formula (I) as defined in claim 1, an enantiomer, a diastereoisomer, hydrate, a solvate, a crystal form, a tautomer, or a pharmaceutically acceptable salt; and
a peptide or a nanostructure;
the peptide having:
a length of 25 amino acids or less and comprising the sequence X₁—X₂—X₃—X₄—X₅—X₆—X₇—X₈—X₉—X₁₀—X₁₁—X₁₂—X₁₃ (SEQ ID NO: 1), wherein X₁ is Leu, Val or Ile, or a non-natural or natural analogue thereof, X₂ is Val, or a non-natural or natural analogue thereof, X₃ is Val, or a non-natural or natural analogue thereof, X₄ is a natural or non-natural amino acid, X₅ is a natural or non-natural amino acid, X₆ is a natural or non-natural amino acid, X₇ is Tyr, or a non-natural or natural analogue thereof, X₈ is Leu, Val, or Ile, or a non-natural or natural analogue thereof, X₉ is Pro, or a non-natural or natural analogue thereof, X₁₀ is a natural or non-natural amino acid, X₁₁ is a natural or non-natural amino acid, X₁₂ is a natural or non-natural amino acid, X₁₃ is Asp or Glu, or a non-natural or natural analogue thereof, and wherein at least one amino acid in the sequence pursuant to SEQ ID No: 1 is different from the amino acid in the sequence LVVSTTYLPHYFD (SEQ ID No: 3) at the corresponding position,
or a peptidomimetic or retro-inverso peptide thereof, or
a length of 25 amino acids or less and comprising the sequence X₁—X₂—X₃—X₄—X₅—X₆—X₇—X₈—X₉—X₁₀—X₁₁—X₁₂—X₁₃ (SEQ ID NO: 2), wherein X₁ is Leu, Val or Ile, or a non-natural or natural analogue thereof, X₂ is Val, or a non-natural or natural analogue thereof, X₃ is Val, or a non-natural or natural analogue thereof, X₄ is a natural or non-natural amino acid, X₅ is a natural or non-natural amino acid, X₆ is a natural or non-natural amino acid, X₇ is Tyr, or a non-natural or natural analogue thereof, X₈ is Leu, Val, or Ile, or a non-natural or natural analogue thereof, X₉ is Pro, or a non-natural or natural analogue thereof, X₁₀ is a natural or non-natural amino acid, X₁₁ is a natural or non-natural amino acid, X₁₂ is a natural or non-natural amino acid, X₁₃ is Asp or Glu, or a non-natural or natural analogue thereof, X₁₄ is a natural or non-natural amino acid, and wherein at least one amino acid in the sequence pursuant to SEQ ID No: 2 is different from the amino acid in the sequence LVVSTTYLPHYFDN (SEQ ID No: 5) at the corresponding position,
or a peptidomimetic or retro-inverso peptide thereof,
the nanostructure comprising:
a) a nucleic acid scaffold; and
b) at least two peptide moieties, wherein the sequence of each of the at least two peptide moieties is independently selected from the sequence of a peptide as specified above, wherein the nucleic acid scaffold is selected from the group of:
i) a linear nucleic acid scaffold, wherein the at least two peptide moieties are each attached at or near different ends of the nucleic acid scaffold; and
ii) a branched nucleic acid scaffold, wherein the at least two peptide moieties are each attached to a different branch of the scaffold.

16. The compound or pharmaceutical composition according to claim 14, wherein:
R is selected from optionally substituted C₁₋₆ alkyl, NR⁷R⁸, and 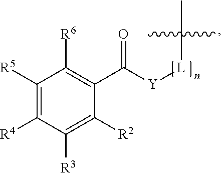
wherein n is 1; and
R¹, R⁷ and R⁸ are defined according to the following (a) or (b):
(a) R¹ is optionally substituted C₁₋₆ alkyl, and R⁷ and R⁸ are selected, independently for each occurrence, from H and optionally substituted C₁₋₆ alkyl; or
(b) R¹ is selected from optionally substituted C₁₋₆ alkyl and NR⁷R⁸, and R⁷ and R⁸ are, independently for each occurrence, optionally substituted C₁₋₆ alkyl.

17. The compound or the pharmaceutical composition according to claim 14, wherein the compound has a structure according any one of the following formulae, an enantiomer, a diastereoisomer, hydrate, a solvate, a crystal form, a tautomer, or a pharmaceutically acceptable salt thereof: [chemical structure]
